(19) United States
(12) Patent Application Publication
(10) Pub. No.: US 2020/0375880 A1
(43) Pub. Date: Dec. 3, 2020

(54) PLANT EXTRACTS FROM THE TAGETES GENUS AND USES OF SAME

(71) Applicant: PLANT ADVANCED TECHNOLOGIES PAT, Vandoeuvre-les-Nancy (FR)

(72) Inventors: Jean-François GINGLINGER, Strasbourg (FR); Simon DESLIS, Saint-Hélen (FR); Léna EMILE, Clission (FR)

(21) Appl. No.: 16/463,956

(22) PCT Filed: Nov. 23, 2017
(86) PCT No.: PCT/EP2017/080224

§ 371 Date: May 24, 2019

(30) Foreign Application Priority Data
Nov. 25, 2016 (FR) 1661486

(51) Int. Cl. A61K 8/9789; A61K 36/28; A61K 8/37; A61K 31/202; A61Q 19/08; A01G 31/00; A01G 22/60

(52) U.S. Cl. CPC A61K 8/9789; A61K 36/28; A61K 8/375; A61K 2236/331; A61Q 19/08; A01G 31/00; A01G 22/60; A61K 31/202

(57) ABSTRACT

An extract of plants from the Tagetes genus enriched with leontopodic acid B, a method for preparing the extract, a cosmetic composition, a nutraceutical composition, and a pharmaceutical composition, where the compositions includes, as the active agent, at least one extract of plants from the Tagetes genus. A method of using the extract as a drug for preventing and/or treating a neurodegenerative disease, including Alzheimer's disease. A cosmetic composition including the extract.

### FIELD OF THE INVENTION

The present invention is concerned with the field of plant extracts and relates to a plant extract from the genus Tagetes enriched with leontopodic acid B, a process for preparing such an extract as well as a cosmetic composition and a pharmaceutical or nutraceutical composition, said compositions comprising as an active agent, at least one plant extracts from the genus Tagetes according to the invention. Another object of the invention is the use of such a plant extract from the genus Tagetes, in particular as a drug for preventing and/or treating a neurodegenerative disease, in particular Alzheimer disease.

### PRIOR ART

Most of the beneficial properties of Edelweiss (Leontopodium alpinum Cass., Asteraceae family) have been ascribed to polyphenolic secondary metabolites, and in particular to leontopodic acid identified as one of the main compounds of the aerial parts of Edelweiss (S. Schwaiger, et al., Leontopodic acid—a novel highly substituted glucaric acid derivative from Edelweiss (Leontopodium alpinum Cass.) and its antioxidative and DNA protecting properties, Tetahedron, Volume 61, Issue 19, 2005, 4621-4630). Leontopodic acid B has been found in a lesser amount than leontopodic acid A in the aerial parts of Edelweiss (S. Schwaiger, et al. Development of an HPLC-PAD-MS assay for the identification and quantification of major phenolic edelweiss (Leontopodium alpium Cass.) constituents, Phytochemical Analysis, volume 17, nº 5, 2006, 291-298). This class of glucaric acid-derived secondary metabolites has rarely been found apart from the genus Leontopodium (S. Schwaiger et al., 2005).

Application CN101856347 describes a pharmaceutical composition containing leontopodic acid extracted from a plant of the genus Edelweiss, used in the preparation of drugs for treating hepatitis.

Costa et al. (2010) teaches that the pre-treatment of cells with leontopodic acid isolated from a plant of the genus Edelweiss causes the decrease in the production of reactive oxygen species (ROS) but induces no improvement in the cell survival (S. Costa et al.; Free radicals and antioxidants in two oxidative-stress cell models exposed to ochratoxin A and amyloid 0: unexpected results, World Mycotoxin journal, August 2010, 3(3): 257-261). The anti-inflammatory effect of Edelweiss ethanolic extracts comprising leontopodic acid has also been described (Lulli D. et al., Anti-inflammatory effects of concentrated ethanol extracts of Edelweiss (Leontopodium alpinum cass.) callus structures towards human keratinocytes and endothelial cells, Mediators of Inflammation, vol. 2012, article ID 498373).

The antioxidant effect exerted by hexaric acid derivatives on neutrophiles is also known (Dudek et al., Caffeic acid derivatives isolated from the aerial parts of Galinsoga parviflora and their effect on inhibiting oxidative burst in human neutrophils, Phytochemistry Letters 16 (2016), 303-310). Edelweiss is a plant dispersed in the high mountains of Europe and Asia at 1 800-3 000 meters of altitude, in inaccessible zones, and it is protected in many countries. It is thus strongly desirable to have new sources of this powerful antioxidant.

Tagetes is a genus of herbaceous plants of the Asteraceae family according to the conventional classification, originating from Mexico, Central America and Western South America. This genus is comprised of about thirty species some of which are cultured as ornamental (Tagetes erecta) or medicinal (Tagetes patula) plants and are used in traditional medicine for their antibacterial, antifungal and soothing properties. The aerial parts of plants from the genus Tagetes are known for their medical or cosmetic properties, in particular via the presence of many phenolic compounds. However, none of the published studies has reported the presence of leontopodic acid in plants from the genus Tagetes.

The inventors have now developed a process enabling a plant extract of Tagetes enriched with leontopodic acid B (ALB) to be prepared. The inventors have actually shown that culturing plants from the genus Tagetes, in particular Tagetes patula and Tagetes erecta, under particular conditions, enables an extract of said plants comprising unexpectedly a very strong content of leontopodic acid B (ALB), detected for the first time in the genus Tagetes, to be obtained. Such a process enables successive extractions to be made without destroying nor worsening the plant survival. The inventors have also shown that a plant extract according to the invention has not only cosmetic benefits, in particular for treating or preventing skin ageing and skin inflammation, but also an interesting neuro-protective activity for preventing or treating neurodegenerative diseases, and more particularly Alzheimer disease.

### DESCRIPTION OF THE FIGURES

FIG. 1 shows a chromatogram of the root extract of Tagetes erecta obtained according to example 1.

FIG. 2 shows a chromatogram of the root extract of Tagetes patula obtained according to example 2.

FIG. 3 shows the survival, in percent of living neurones with respect to the control, of primary cortical neurons intoxicated for 24 h by peptide Aβ1-42 added at a final concentration of 20 μM, in the presence of a root extract from Tagetes erecta at concentration ranging from 10 ng/mL to 10 μg/mL, in accordance with example 5.

The histogram bars represent: control (1); +peptide Aβ1-42 (2); peptide Aβ1-42+root extract of Tagetes erecta 10 nM (3); peptide Aβ1-42+root extract of Tagetes erecta 100 nM (4); peptide Aβ1-42+root extract of Tagetes erecta 1 μM (5); peptide Aβ1-42+root extract of Tagetes erecta 10 μM (6).

FIG. 4 shows the growth of the network of neurites intoxicated for 24 h by the peptide Aβ1-42 added at a final concentration of 20 μM, in the presence of a root extract of Tagetes erecta at concentrations ranging from 10 ng/mL to 10 μg/mL, in accordance with example 5.

The histogram bars represent: control (1), +peptide Aβ1-42 (2), peptide Aβ1-42+root extract of Tagetes erecta 10 nM (3), peptide Aβ1-42+root extract of Tagetes erecta 100 nM (4), peptide Aβ1-42+root extract of Tagetes erecta 1 μM (5), peptide Aβ1-42+root extract of Tagetes erecta 10 μM (6).

### DETAILED DESCRIPTION OF THE INVENTION

A first object of the present invention is a plant extract from the genus Tagetes characterised in that it is enriched with leontopodic acid B and in that it comprises at least 2.5%, in particular at least 5.5%, more particularly at least 8% by weight of leontopodic acid B, expressed relative to the total weight of the dry extract.

More particularly, the object of the present invention is a plant extract from the genus Tagetes enriched with leontopodic acid B and comprising at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, or at least 10% by weight of leontopodic acid B, expressed relative to the total weight of the dry extract.

By “plant extract from the genus Tagetes”, it is meant the product obtained by extracting plants, or a plant, from the genus Tagetes or “marigold”.

By “enriched with leontopodic acid B”, it is meant a plant extract comprising a higher amount of leontopodic acid B, with respect to a corresponding plant extract that can be found in nature.

By “leontopodic acid B” it is meant a compound having the following general formula (I):

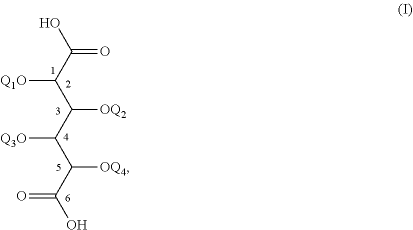

in which any three of the radicals Q₁, Q₂, Q₃ and Q₄ represent a cafeoyl group and the fourth radical represents a hydrogen atom or a 3-hydroxybutanoyl group of the formula CH₃—CH(OH)—CH₂—CO—.

By “leontopodic acid B” or “ALB”, it is meant a compound having the general formula (I) in which any three of the radicals Q₁, Q₂, Q₃ and Q₄ represent a cafeoyl group and the fourth radical represents a hydrogen atom.

By “leontopodic acid B1” or “ALB1”, it is intended a compound of the formula (I) in which Q₂, Q₃ and Q₄ represent a cafeoyl group and Q₁ represents a hydrogen atom.

By “leontopodic acid B2” or “ALB2”, it is intended a compound of the formula (I) in which any three of the radicals Q₁, Q₂, Q₃ and Q₄ represent a cafeoyl group and the fourth radical represents a hydrogen atom, said compound being characterised in that the mass m/z of the ionised product (otherwise called a molecular ion) in negative mode mass spectrometry is 695 and in that its chromatographic retention time is higher than that of leontopodic acid B1 under the experimental conditions described in example 1.1.

By “leontopodic acid B3” or “ALB3”, it is intended a compound of the formula (I) in which any three of the radicals Q₁, Q₂, Q₃ and Q₄ represent a cafeoyl group and the fourth radical represents a hydrogen atom, said compound being characterised in that the mass m/z of the ionised product (otherwise called a molecular ion) in negative mode mass spectrometry is 695 and in that its chromatographic retention time is higher than that of leontopodic acid B2 under the experimental conditions described in example 1.1.

By “leontopodic acid A”, or “ALA”, it is intended a compound having the general formula (I) in which any three of the radicals Q₁, Q₂, Q₃ and Q₄ represent a cafeoyl group and the fourth radical represents a 3-hydroxybutanoyl group of the formula CH₃—CH(OH)—CH₂—CO—.

More particularly, by “leontopodic acid A”, it is meant a compound of the formula (I) in which Q₂, Q₃ and Q₄ represent a cafeoyl group and Q₁ represents a group CH₃—CH(OH)—CH₂CO—.

The different position isomers of ALB, in particular ALB1, ALB2 and ALB3, and ALA are thus acids of the general formula (I) substituted with 3 cafeoyl groups and with a radical R, with R chosen from: a 3-hydroxybutanoyl group and a hydrogen atom, as represented in Table 1 below:

TABLE 1
Structure of leontopodic acids
Leontopodic acid (ALA or ALB)

| Q₁ | Q₂ | Q₃ | Q₄ |
|---|---|---|---|
| R | Cafeoyl | Cafeoyl | Cafeoyl |
| Cafeoyl | R | Cafeoyl | Cafeoyl |
| Cafeoyl | Cafeoyl | R | Cafeoyl |
| Cafeoyl | Cafeoyl | Cafeoyl | R |

By “dry extract”, it is meant the extract obtained by implementing the process followed by a step of drying the extract, the drying being implemented according to any method well known to those skilled in the art, in particular placing the extract in a hot and dry atmosphere. By “dry root biomass”, it is meant the dry root biomass determined before conducting the root extracting step. For the solid/liquid extraction from fresh root, the dry root biomass has been measured at 5±1.5% of the fresh root biomass.

According to an advantageous aspect, an extract according to the present invention can be also defined in that it comprises at least 0.75% by weight of leontopodic acid B, expressed relative to the total dry biomass weight, more preferentially at least 1.5%, at least 2%, at least 2.5%, or at least 3% by weight of leontopodic acid B.

According to a particular aspect, an extract according to the invention is characterised in that said leontopodic acid B comprises the position isomers ALB1, ALB2 and ALB3, said isomer ALB2 being present in a proportion of at least 50%, preferentially at least 80% of the total weight of the leontopodic acid B present in said extract.

According to another particular aspect, an extract according to the invention is characterised in that it does not comprise leontopodic acid A in a detectable amount, or in that it comprises a low or a very low amount leontopodic acid A, and preferentially less than 2%, preferably less than 1.5%, more preferentially less than 1% and even preferentially less than 0.5% of leontopodic acid A, in percent of the total weight of leontopodic acid present in said extract.

According to another advantageous aspect, an extract according to the present invention comprises at least 2.5% by weight of leontopodic acid B, expressed with respect to the total weight of the dry extract, more preferentially at least 5.5%, at least 6.5%, at least 7%, at least 7.5%, at least 8% and at least 8.5% by weight of leontopodic acid B expressed relative to the total weight of the dry extract, and comprises less than 1.5%, preferably less than 1%, more preferentially less than 0.5% of leontopodic acid A, in percent of the total weight of the leontopodic acid present in said extract.

According to another particular aspect, a plant extract from the genus Tagetes according to the invention is an extract from a Tagetes species chosen from Tagetes erecta and Tagetes patula.

According to another particular aspect, a plant extract from the genus Tagetes according to the invention is an extract of a part of the plant, that is a constituent part of a plant such as the roots, stem, leaves, blossom or seed. Advantageously, one object of the invention is a root extract of a plant from the genus Tagetes.

For obtaining an extract of the different aerial parts, when the same are considered sufficiently developed, they are contacted with a leaching liquid or solvent by percolation, sprinkling, dipping or maceration and then said leaching liquid is recovered and treated to extract the molecules of interest therefrom which have been released by the aerial parts of said plants. For obtaining a root extract, when the roots are considered sufficiently developed, the same are contacted with a solvent by dipping or maceration, and then said solvent is recovered and treated to extract the molecules of interest therefrom which have been released by the roots of said plants. This type of process corresponds partly to the process developed by The Company Plant Advanced Technologies (PAT) as “PAT Plantes a Traire®” and described in the international application WO 01/33942. The content of the international application WO 01/33942 is incorporated in reference to the present description.

According to a particular aspect, one object of the invention is a plant extractfrom the genus Tagetes, in particular a root extract, more particularly chosen from:
an extract obtained by root exudation in a solvent,
an extract obtained by root maceration in a solvent, and
a mixture of at least one extract obtained by root exudation in a first solvent and at least one extract obtained by root maceration in a second solvent.

In a second aspect, one object of the invention is a process for preparing a plant extractfrom the genus Tagetes according to the invention comprising:

a) a step of culturing Tagetes under soil-less conditions, and in particular in aeroponics,

b) optionally a step of stimulating the plants,

c) a step of solid/liquid extracting the plants optionally stimulated during step b), and

d) recovering the extract obtained during step c).

According to a particular embodiment of this second aspect, one object of the invention is a process for preparing a plant extract from the genus Tagetes according to the invention, comprising:

a) a step of culturing Tagetes under soil-less conditions, and in particular in aeroponics,

b) optionally a step of stimulating the plant roots,

c) a step of solid/liquid extraction of the roots optionally stimulated during step b), and

d) recovering the extract obtained during step c).

Within the scope of the invention, by “culturing plants under soil-less conditions”, it is meant any culture mode in which the plant roots are not in the soil. More precisely, the soil-less culture is a culture in which the plant roots lay in a reconstituted medium, detached from the soil. This culture medium is regularly irrigated by nutrient solutions suitable for the cultured plant.

There are different soil-less culture techniques such as substrateless systems which require an oxygen-enriched nutrient solution, and systems with a substrate. Among substrateless systems, aquiculture for which the nutrient solution is not circulating and is contained in a culture vessel, Nutrient Film Technique (N.F.T.) for which the nutrient solution is enriched with oxygen dissolved during its displacement by air exchange, and aeroponics for which the plant roots are in contact neither with a solid medium, nor with a liquid medium: they are fed by a nutrient mist obtained by atomising (via an atomiser) of the nutrient solution in a closed medium, can be mentioned. Among the systems with a substrate, there are the subirrigation in which the nutrient solution penetrates the substrate at its lower part and percolation in which the nutrient solution is distributed by batch irrigation at the upper surface of the system and then percolates downwardly of the substrate. The mineral or organic substrate is neutral and inert like sand, clay or rock wool for example. This substrate can also be of industrial origin.

By “culturing the plants in aeroponics”, it is meant a soil-less culture mode for which the plant roots are in contact neither with a solid medium, nor with a liquid medium. According to a particular embodiment, the plants are fed by a nutrient mist obtained by atomising, via an atomiser, the nutrient solution in a closed medium.

Typically, in a process according to the invention, plants can be disposed on trays with the aerial part of the plant on top of the tray and the root part therebelow, the trays are disposed on tables forming a retention zone to collect the excess of a liquid diffused to the plants, and the trays are transferred onto the tables at different stations.

During step a), the plants cultured in aeroponics are fed by root spraying a nutrient solution of essential mineral salts (Nitrogen—N, Phosphorus—P, Potassium—K), in order to obtain a maximum root development and a maximum concentration of molecules of interest, without worsening the plant survival. Those skilled in the art, using their general knowledge, will be able to know how to adapt the proportions and concentrations of different mineral salts to optimise root development and concentration of the molecules of interest. The concentrations of mineral salts of the nutrient solutions are in this case in a low electroconductivity range advantageously ranging from 0.4 to 1.6 mS/cm, preferably between 0.6 to 0.8 mS, in order to promote a wider diversity of molecules in the extracts.

In step c) of a process according to the invention, by “solid/liquid extraction”, it is meant any solvent extraction technique which consists in extracting a chemical species being in a solid and being soluble in said solvent. Among these extraction techniques, maceration, exudation, infusion, decoction, extraction through Soxhlet and Kumagawa extractors, microwave assisted extraction, ultrasound assisted extraction, extraction through the enzyme pathway, supercritical fluid extraction (CO2+DPG) can be mentioned. Extraction leads to the recovery of the metabolites contained in either part of the plants, and in particular the roots, by means of a leaching liquid, or solvent, put in contact with the cut off roots and/or the roots still bound to the plant, in a particular solvent and for an appropriate duration.

According to a particular aspect, in a process according to the invention, step c) of solid/liquid extraction of the plants or a part of the plants, is made by means of a process chosen from: exudation and maceration.

According to a more particular aspect, in a process according to the invention, step c) of solid/liquid extraction of the plants or a part of the plants, advantageously the roots potentially stimulated during step b), is made by means of a process chosen from: root exudation and root maceration. This step consists in particular of a “soaking” step, during which the plant roots are put into a soaking vessel containing a liquid for a predetermined duration. Typically, the plants are disposed on a tray with the aerial part of the plant above the tray and the root part therebelow, the tray is lifted after soaking, and then the liquid contained in the soaking vessel is collected. According to a particular aspect, the soaking step is followed by a cutting step during which the aerial part or the root part of the plants is partially cut off to collect the cut off part. It is possible to extract the substances of the cut off parts, in addition to the extraction of the substances upon soaking.

Preferably, in a process according to the invention, the root exudation is made on roots still fixed to the plant. According to another preferred aspect, the root maceration is made on freshly cut roots, that is roots cut within 24 hours, and preferably as soon as possible after cutting, ideally just after cutting, said roots being possibly dried after being severed and before maceration. The root drying can be made by implementing any adapted drying process, known to those skilled in the art, and in particular by placing the roots at a temperature between 40° C. and 60° C. for 4 hours, preferably in a dry environment. The root drying can in particular be made in a ventilated stove.

More particularly, step c) of a process according to the invention comprises bringing the roots in contact with a solvent chosen from: alcohols, glycols and eutectic solvents. Said alcohol is preferably chosen from ethanol and methanol, used pure or in the form of an aqueous solution of alcohol, the same comprising from 10% to 99.9% alcohol, more particularly between 40% and 90%, and further particularly between 50% and 85%. Said glycol is a diol in which both hydroxyl groups are carried by different carbons, and is chosen from: dipropylene glycol, propane 1,3 diol, propane 1,2 diol and butylene glycol, and is used pure or in the form of an aqueous solution of glycol, the same comprising from 10% to 99.9% of glycol, more particularly between 40% and 90%, and further particularly between 50% and 85%. Said eutectic solvent consists of two or more components, which can be solid or liquid and which, in a particular composition, have a strong decrease in their melting temperature, thus making the mixture liquid. The natural eutectic solvents mainly consist of amino acids, organic acids, sugars and choline derivatives. Water can be part of the solvent, but is in this case strongly retained in the liquid and cannot be evaporated. The particular combinations of the components making up said eutectic solvent are chosen in particular from, and in a non-exhaustive way, choline chloride—glucose (ratio 1:1), choline chloride—citric acid (ratio 1:1), choline chloride citric acid (ratio 2:1), choline chloride—sucrose (ratio 4:1), choline chloride—sucrose (ratio 1:1), choline chloride—tartaric acid (ratio 2:1), choline chloride—xylose (ratio 2:1), choline chloride—xylose (ratio 3:1), citric acid—sucrose (ratio 1:1), citric acid—glucose (ratio 1:1), glucose—tartaric acid (ratio 1:1), choline chloride—urea (ratio 1:2), choline chloride—xylitol—water (2:1:3) and lactic acid—glucose—water (5:1:3).

These solvents enable the extraction of a strong content of leontopodic acid B (ALB) and its isomers to be promoted. In the case of a mixture of root extracts, said first and second solvents can be identical or different.

According to a particular aspect of the invention, the solvent is characterised by an acidic pH, in particular when used during the solid/liquid extraction step. According to a particular aspect, said alcohol or said glycol used during a solid/liquid extraction step is characterised by a pH between 2 and 5, preferably between and 4, more preferentially between 2.5 and 3.5. According to another particular aspect, a plant extract according to the invention is characterised by a pH between 2.5 and 5, preferably between 2.5 and 4 and more advantageously between 3 and 4. The use of a solvent with an acidic pH promotes the solubilisation of the compounds of interest in said solvent and limits the chemical or enzymatic degradation of the secondary metabolites including ALB during extraction. The acids used for adjusting the pH of the solvent or the extract are preferably phosphoric acid, citric acid or hydrochloric acid.

More particularly, said glycol is chosen from the following group: dipropylene glycol, propane 1,3 diol, propane 1,2 diol and butylene glycol.

Propane 1,3 diol, or trimethylene glycol, can be prepared by chemical synthesis according to techniques known to those skilled in the art and described in literature, it is also available in the market. Said propane 1,3 diol can also be produced by implementing a fermentation process under adapted conditions of a living organism, in particular a genetically modified strain of Escherichia coli. The propane 1,3 diol thus obtained is designated by the term “biosourced propane 1,3 diol”, such a product is produced and then purified as described in particular in the international application WO 2004/101479 and marketed as the brand Zéméa®. By “butylene glycol”, it is meant butane 1,2 diol, butane 1,3 diol, butane 2,3 diol and butane 1,4 diol.

Even more particularly, in a process according to the invention, step c) of solid/liquid extraction comprises bringing the roots in contact with propane 1,2 diol or with propane 1,3 diol, and in particular biosourced propane 1,3 diol.

According to a particular aspect, step c) of a process according to the invention comprises bringing the roots in contact with a solvent for a duration between 15 minutes and 30 minutes for root exudation and for a duration between 1 hour and 96 hours, in particular between 24 hours and 72 hours, more particularly between 36 hours and 48 hours for root maceration.

According to another particular aspect, a process according to the invention comprises a step of severing the roots and then drying the severed roots, prior to the step of macerating said roots, the maceration being made by bringing the roots in contact with a solvent.

The process according to the invention can thus comprise a first root exudation step performed by dipping, in a solvent, roots still bound to the plant for a duration between 15 minutes and 30 minutes, followed by a root maceration step of the severed roots of the plant, and then possibly dried.

The root exudation and/or root maceration durations are chosen so as to promote extraction of a high amount of compounds of interest while not worsening the plant survival and without leading to the resource depletion. Thus, the plants, after the root exudation step, are re-cultured in aeroponics according to steps a) and possibly b) in order to restart their root development and promote production by the roots of secondary metabolites.

According to a particular aspect, a process according to the invention is a process of preparing a root extract from the genus Tagetes.

According to a preferred aspect, a process according to the invention is a process of preparing a root extract from the genus Tagetes erecta or Tagetes Patula.

According to a preferred embodiment, another object of the invention is a process for preparing a plant extract from the genus Tagetes according to the invention comprising:

a) a step of culturing Tagetes under soil-less conditions, and in particular in aeroponics,

b) a step of stimulating the plant roots,

c) a step of solid/liquid extraction of the roots stimulated during step b), and

d) recovering the extract obtained during step c).

According to another particular aspect, in a process according to the invention, step b) of stimulating the plant roots comprises:
an elicitation step in which the roots are brought in contact with a solution comprising at least one agent chosen from: a salt, a surfactant, a solvent, a fungal or bacterial origin elicitor, a jasmonic acid derivative, in particular methyl jasmonate, salicylic acid, an ethylene generator, a chitine, chitosans and/or mixture thereof, and/or
a step of bringing the roots in contact with a nitrogen deficient solution, that is a solution comprising a nitrogen proportion lower than the nitrogen proportion usually considered as optimum for the plant and in particular the root development, advantageously less than 15% of nitrogen, and advantageously comprising no nitrogen, said steps being sequential or simultaneous.

The roots can also be stimulated by bringing the plants in contact with a nitrogen deficient nutrient solution. Bringing the plants in contact with a nitrogen deficient nutrient solution causes a “nitrogen stress” responsible for the stimulation. According to a particular aspect, a nitrogen deficient solution according to the present invention is a solution comprising less than 15% of nitrogen, preferably less than 10% of nitrogen, advantageously less than 8%, more advantageously less than 6%, less than 5%, less than 4%, less than 3%, less than 2%, less than 1% of nitrogen and even more advantageously 0% of nitrogen.

The stimulation step b) enables the content of secondary metabolites in the roots to be significantly increased and thus the flux of the metabolites exiting the roots to the solvent chosen for extraction to be promoted, without total loss for the plant viability such that it can be re-cultured and then reused. In other words, the step of stimulating the plant enables production and secretion of the molecules of interest to be favoured.

Advantageously, step b) of stimulating the roots is made by spraying or macerating the plant with a solution of elicitors chosen from jasmonic acid derivatives as for example methyl jasmonate, salicylic acid and chitosans or by feeding the plant with a nitrogen deficient N/P/K nutrient solution vaporised on the roots.

According to a particular embodiment of the invention, step b) is implemented by spraying or macerating the plant with a solution chosen from:
a methyl jasmonate solution at a concentration between 0.01 and 200 μM, advantageously between 0.1 and 20 μM,
a salicylic acid solution at a concentration between 1 and 500 μM, advantageously between 10 and 50 μM,
a chitosan solution at a concentration of 0.002 to 1 g/L, advantageously of 0.05 to 0.1 g/L, and
a N/P/K nutrient solution comprising less than 6% of nitrogen, said solution being preferably vaporised on the roots.

Further, step b) of stimulating the roots with an elicitor solution is advantageously performed for a duration between 1 day and 21 days, preferably between 1 and 7 days.

According to a particular aspect, step b) of stimulating the roots by feeding the plant with a nitrogen deficient N/P/K nutrient solution vaporised on the roots is advantageously performed for a duration between 10 days and 8 weeks, preferably 3 weeks.

During step b), the concentrations of mineral salts of the nutrient solutions are included in a low electroconductivity range advantageously extending between 0.4 and 1.6 mS/cm, preferably between 0.6 and 0.8 mS, in order to favour a wider diversity of molecules in the extracts.

According to a particular aspect of a process according to the invention, step b) is made after step a). According to another particular aspect of a process according to the invention, step b) and step a) are performed simultaneously, the stimulation solution is therefore incorporated to the nutrient solution or administrated by any other administration mode known to those skilled in the art.

According to an improvement, the “soaking” step is preceded with a washing step in which the liquid diffused to the plants is clear water. Thus, the supply of elements contained in the nutrient solution or in the possible stimulation solution is limited during the soaking step.

Advantageously, one object of the invention is a process comprising the following steps:

a) a step of culturing Tagetes under soil-less conditions, and in particular in aeroponics,

b) a step of stimulating the plant roots,

c) a step of solid/liquid extraction of the roots stimulated during step b), comprising:
a first phase of bringing the roots still attached to the plant in contact with a solvent, in particular for a duration between 15 minutes and 30 minutes,
followed by a second phase comprising severing said roots and macerating the severed roots in a solvent,

said solvent, identical or different in the first and the second phase, is chosen from alcohols, glycols and eutectic solvents, and being used pure or in the form of an aqueous solution of alcohol, glycol or eutectic solvent, and

d) recovering the extracts obtained during step c).

A process according to the invention advantageously comprises a further step of readjusting the solvent content of the extract, to obtain an advantageous content of at least 50%, and/or adjusting the pH of the extract, to obtain a pH advantageously between 3 and 5, preferably of around 3.5, for preserving the extract.

A process according to the invention advantageously comprises at least one further step of purifying and/or treating the plant extract, such step being chosen in particular from:
at least one filtration, in particular a nanofiltration and/or a sterilising filtration and/or a clarifying filtration,
a liquid/solid extraction,
a purification,
a concentration and
a discoloration of the root extract,
   such purification and/or treatment methods are well known to those skilled in the art. This step enables the final extract of Tagetes to be obtained, preferably of Tagetes patula and Tagetes erecta and in particular of Tagetes erecta rich with polyphenols, in particular with leontopodic B (ALB) and its position isomers.

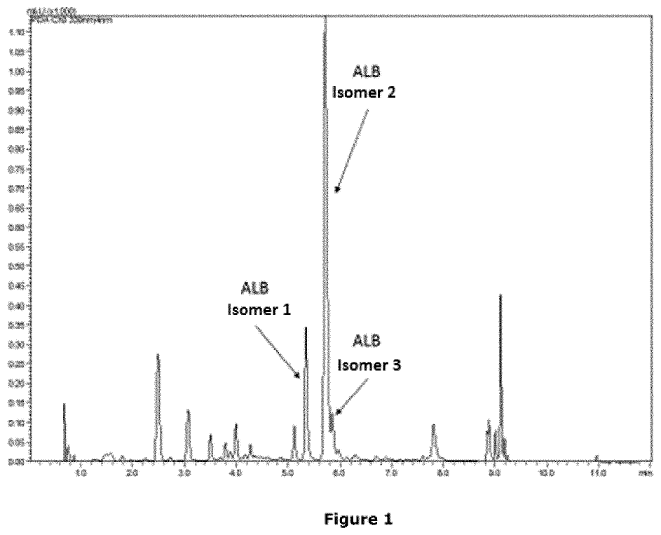

The process according to the invention enables a plant extractfrom the genus Tagetes preferably Tagetes patula and Tagetes erecta to be obtained, which is particularly rich with polyphenols, in particular with leontopodic acid B and its isomers. In particular, the plant extract obtained by the process described above, is rich with a mixture of the different isomers of ALB, for example ALB1, ALB2 and ALB3. A chromatogram (FIG. 1) of the analysis of a root extract of Tagetes erecta obtained according to the invention, shows that leontopodic acid B is present in this extract in the form of at least three isomers: ALB1, ALB2 and ALB3. ALB2 is the isomer mostly present. Preferably, at least 50%, still more preferentially at least 80% by weight of said ALBs are in the form of the isomer ALB2, relative to the total weight of leontopodic acid B.

Another object of the present invention is an extract likely to be obtained by a process according to the invention.

A third object of the present invention is a cosmetic composition comprising, as an active agent, at least one extract according to the invention or an extract obtained by a process according to the invention, and at least one excipient. Advantageously, said extract is a plant extract from the genus Tagetes, preferably Tagetes patula and Tagetes erecta and in particular a root extract from the genus Tagetes, in particular a root extract from Tagetes patula or Tagetes erecta according to the invention.

The administration modes, dosages and optimum dosage forms of a cosmetic composition according to the invention can be determined according to the criteria generally considered in establishing a cosmetic treatment adapted to a subject such as for example the skin type.

The cosmetic composition according to the invention is advantageously intended to a topical application. It can be in particular in the form of a cream, milk, lotion, gel, serum, spray, foam, solution, ointment, emulsion, patch or mask. A cosmetic composition according to the invention comprises at least one cosmetically acceptable excipient chosen as a function of the administration type desired. A cosmetic composition according to the present invention can further comprise at least one adjuvant known to those skilled in the art, chosen from thickeners, preservatives, fragrances, colorants, chemical or mineral filters, moisturising agents, thermal waters, etc.

A cosmetically acceptable excipient can be chosen from polymers, silicone compounds, surfactants, rheology agents, humectant agents, penetration agents, oil components, waxes, emulsifiers, film forming agents, fragrances, electrolytes, pH adjusters, antioxidant agents, preservatives, colorants, pearls, pigments and mixtures thereof.

A cosmetic composition according to the invention can further comprise at least one other cosmetically active agent, such as another anti-age agent, a moisturising agent, an agent having a calming, soothing or relaxing activity, an agent stimulating skin microcirculation, a sebo-regulator agent for oily skin care, a cleaning or purifying agent, an anti-radical agent, an anti-inflammatory agent, a chemical or mineral solar screen, etc.

Advantageously, a cosmetic composition according to the invention comprises at least one extract from Tagetes according to the invention, and in particular a root extract from Tagetes patula or Tagetes erecta according to the invention, in an amount between 0.01 and 10%, in particular 0.05 and 5%, more particularly between 0.1 and 2%, by weight relative to the total weight of the composition.

A fourth object of the present invention is an extract according to the invention, or an extract obtained by a process according to the invention, or a cosmetic composition according to the invention, for preventing or slowing skin ageing, and/or having a skin soothing or calming effect following the irritation feeling, and/or stimulating epidermal turnover as well as epidermal barrier function, and skin moisturising, and/or for protecting the epidermis from environmental stresses.

A cosmetic composition according to the invention can in particular be for preventing or slowing skin ageing. A cosmetic composition according to the invention can also in particular be for having a skin soothing or calming effect following the irritation feeling which can be developed after shaving, friction, or contact with allergens. A cosmetic composition according to the invention can in particular be for promoting integrity and efficiency of the epidermal barrier function. A cosmetic composition according to the invention can in particular be for stimulating skin moisturising.

A fifth object of the present invention is a pharmaceutical composition comprising, as an active agent, at least one extract according to the invention or an extract obtained by a process according to the invention, and at least one pharmaceutically acceptable excipient, and a nutraceutical composition comprising, as an active agent, at least one extract according to the invention or an extract obtained by a process according to the invention, and at least one nutraceutically acceptable excipient.

In the present invention, by “pharmaceutically or nutraceutically acceptable”, it is meant any ingredient which is useful in preparing a pharmaceutical or nutraceutical composition, which is generally safe, non-toxic and neither biologically nor otherwise undesirable and which is acceptable for a veterinary use or in humans.

By “pharmaceutically or nutraceutically acceptable salts” of a compound, it is meant salts which are pharmaceutically or nutraceutically acceptable, as defined above, and which have the desired pharmacological activity of the parent compound. Such salts comprise:

(1) hydrates and solvates,

(2) pharmaceutically or nutraceutically acceptable acid addition salts formed with pharmaceutically or nutraceutically acceptable inorganic acids such as hydrochloric acid, hydrobromic acid, sulphuric acid, nitric acid, phosphoric acid and the like; or formed with pharmaceutically or nutraceutically acceptable organic acids such as acetic acid, benzenesulphonic acid, benzoic acid, camphresulphonic acid, citric acid, ethane-sulphonic acid, fumaric acid, glucoheptonic acid, gluconic acid, glutamic acid, glycolic acid, hydroxynaphthoic acid, 2-hydroxyethanesulphonic acid, lactic acid, maleic acid, malic acid, mandelic acid, methanesulphonic acid, muconic acid, 2-naphthalenesulphonic acid, propionic acid, salicylic acid, succinic acid, dibenzoyl-L-tartaric acid, tartaric acid, p-toluenesulphonic acid, trimethylacetic acid, trifluoroacetic acid and the like, or

(3) pharmaceutically or nutraceutically acceptable base addition salts formed when an acidic proton present in the parent compound is either replaced with a metal ion, for example an alkaline metal ion, an alkaline earth metal ion or an aluminium ion; or coordinated with a pharmaceutically or nutraceutically acceptable organic or inorganic base. The acceptable organic bases comprise diethanolamine, ethanolamine, N-methylglucamine, triethanolamine, tromethamine and the like. The acceptable inorganic bases comprise aluminium hydroxide, calcium hydroxide, potassium hydroxide, sodium carbonate and sodium hydroxide.

The administration modes, dosages and optimum dosage forms of a pharmaceutical composition according to the invention can be determined according to the criteria generally considered in establishing a pharmaceutical treatment adapted to a subject as for example the age or body weight of a patient, his/her general status gravity, treatment tolerance, the secondary effects observed, skin type. As a function of the administration type desired, the pharmaceutical composition according to the invention can further comprise at least one pharmaceutically acceptable excipient. The pharmaceutical composition according to the present invention can further comprise at least one pharmaceutical adjuvant known to those skilled in the art, chosen from thickeners, preservatives, fragrances, colorants, chemical or mineral filters, moisturising agents, thermal waters, etc.

Advantageously, said pharmaceutical composition comprises at least one extract according to the invention in an amount between 0.01 and 10%, in particular between 0.05 and 5%, more particularly between 0.1 and 2%, by weight relative to the total weight of the composition.

A pharmaceutical composition is particularly suitable for an administration through the oral, nasal, transdermal, parenteral, topic, rectal and mucosal way. It can be in dry form, such as for example: a soft capsule, hard gelatine capsule, tablet, lyophilisate, powder, granule, or patch, or in liquid form, such as: solution, suspension, spray, cream or gel.

The pharmaceutically acceptable excipient is known to those skilled in the art and is chosen according to the administration mode of the pharmaceutical composition. By way of example, the pharmaceutically acceptable excipient can be chosen from the group consisting of diluent agents, binders, disintegrants, colorants, lubricants, solubilising agents, absorption promoting agents, film forming agents, gelling agents, and mixtures thereof.

The pharmaceutical composition according to the invention can further comprise at least one compound chosen from the group consisting of emollients, moisturising active agents, keratin synthesis activators, keratoregulators, keratolytics, skin barrier restructuring agents (skin lipid synthesis activators, PPAR agonists or Peroxisome Proliferator Activated Receptor), keratinocyte differentiation activators (retinoids, Calcidone®, calcium), antibiotics, anti-bacterial agents, antifungal compounds, anti-viral agents, sebo-regulators, immunomodulators, such as tacrolimus, pimecrolimus, oxazolines, preservatives, anti-irritating agents, soothing agents, solar filters and screens, antioxidant agents, growth factors, healing agents or eutrophic molecules, anti-inflammatory drugs and agents, anti-Alzheimer drugs and agents.

A sixth object of the present invention is an extract according to the invention or a pharmaceutical composition according to the invention, for use as a drug.

More particularly, another object of the present invention is an extract according to the invention, an extract obtained by a process according to the invention, or a pharmaceutical composition according to the invention, or a nutraceutical composition according to the invention, for use as a drug for stimulating antimicrobial defence, and/or mitigating or treating skin inflammation and/or promoting skin healing, in particular in the case of a wound closure.

More particularly, another object of the present invention is an extract according to the invention, an extract obtained by a process according to the invention, or a pharmaceutical composition according to the invention, for use as a drug for preventing or slowing skin ageing, for having a skin soothing or calming effect following the irritation feeling, for stimulating the epidermal barrier function as well as water moisturising. In this case, said extract will be advantageously associated with a pharmaceutically acceptable excipient and adapted for a skin application, and said pharmaceutical composition will advantageously contain a pharmaceutically acceptable excipient and adapted for a skin application.

According to another particular aspect, another object of the present invention is an extract according to the invention, an extract obtained by a process according to the invention, or a pharmaceutical composition according to the invention or a nutraceutical composition according to the invention, for use as a drug for preventing or treating a neurodegenerative disease.

Within the scope of the present invention, by “neurodegenerative disease”, it is meant a disease mainly involving a deterioration of the operation, and possibly the death, of nerve cells, and in particular neurones. These diseases cause cognitive-behavioural, sensorial and motor type disorders.

According to a particular aspect, said neurodegenerative disease is chosen from: Alzheimer disease, spinocerebellar ataxia, multiple system atrophy, Alexander disease, Alpers disease, Creutzfeldt-Jakob disease, Huntington disease, Parkinson disease, Pick disease, macrophagic myofasciitis, progressive supranuclear palsy, multiple sclerosis and amyotrophic lateral sclerosis.

Within the scope of the present invention, a neurodegenerative disease can be a neurodegenerative disease due to oxidative stress.

It is in particular the case of Alzheimer disease. By “Alzheimer disease” (AD), it is meant a disease mainly affecting people older than 65, suffering from different clinical symptoms such as a progressive impairment of memory, thinking, language and capability of learning. The toxic role of the β-amyloid (Aβ) peptide now switches from insoluble Aβ fibrils to smaller soluble aggregates of Aβ oligomers. It has been set out that soluble Aβ oligomers isolated from cortex of patients having Alzheimer disease directly induce protein Tau (T) hyperphosphorylation and neuritic degenerescence (Jin M, et al., Soluble amyloid beta-protein dimers isolated from Alzheimer disease cortex directly induce Tau hyperphosphorylation and neuritic degenerescence. Proc Natl Acad Sci USA. 2011 Apr. 5; 108(14):5819-24). Thus, all the substances having a property reducing peptide Aβ neurotoxicity can be useful as a new therapeutic agent for treating or preventing Alzheimer disease.

According to a particular aspect, an object of the present invention is therefore an extract according to the invention, an extract obtained by a process according to the invention, or a pharmaceutical composition according to the invention, for use as a drug for preventing or treating Alzheimer disease.

Another object of the present invention is a skin cosmetic care method for preventing or delaying the onset of the skin ageing effects, and/or having a skin soothing or calming effect following the irritation feeling and/or stimulating epidermal turnover as well as the epidermal barrier function and skin moisturising, and/or for protecting the epidermis from environmental stresses, said method being characterised in that it comprises applying, on at least one part of the body or face skin, a cosmetic composition according to the invention.

Advantageously, in the method according to the invention, the cosmetic composition is applied in a subject in need thereof, in particular in anticipation of or following a skin single or repeated exposure to an oxidative stress. Indeed, the latter can generate an excess of free radicals that can accelerate skin ageing signs.

Finally, one object of the invention is a method for preventing and/or treating a neurodegenerative disease, in particular one of the abovementioned diseases, and more particularly Alzheimer disease, comprising administrating a therapeutically efficient amount of at least one compound of an extract according to the invention, an extract prepared by a process according to the invention, a nutraceutical composition according to the invention or a pharmaceutical composition according to the invention to a patient in need thereof.

Further, the invention relates to a method for preventing or slowing skin ageing, for mitigating skin inflammation, for having a skin soothing or calming effect following the irritation feeling, for stimulating the epidermal barrier function as well as skin moisturising, and for promoting skin healing, in particular in the case of a wound closure, in a subject in need thereof, comprising administrating to the subject a therapeutically efficient amount of a nutraceutical composition according to the invention or a pharmaceutical composition according to the invention.

The present invention also relates to a nutraceutical composition comprising an extract according to the invention, or an extract obtained by a process according to the invention, and a nutraceutically acceptable excipient, for improving the cognitive functions in a patient having a neurodegenerative disease, and in particular Alzheimer disease. The nutraceutical acceptable excipient is known to those skilled in the art and is chosen from the excipients in accordance with the international regulations applicable to food additives.

According to a particular aspect, other botanical extracts known to those skilled in the art can also be added to a nutraceutical composition according to the invention to improve the cognitive functions in a patient having a neurodegenerative disease, and in particular Alzheimer disease. These botanical extracts can be chosen from extracts of plants or a part of plants (for example root, stem, leaf, blossom, seed, bud, fruits) as citrus fruits (orange, lemon, grapefruit), the genus Rosa (rose hip, rose tree), Panax ginseng, Bacopa monnieri, black currant, Ginkgo biloba, vine, savory, rosemary, alder, walnut tree, olive tree.

Examples 1 to 4 that follow and FIGS. 1 to 4 aim at illustrating the present invention, however without limiting the scope thereof.

### EXAMPLES

### Example 1: Preparation and Characterisation of Root Extracts of Tagetes erecta According to the Invention

The Tagetes erecta extracts are prepared from plants originating from Reunion Island. The supplier is Societe Horticole de Bassin Plat, EARL Bassin Plat 31, Chemin de la Croix de Jubile 97410 Saint Pierre, Reunion Island.

### 1.1 Extract Prepared from a Glycol Solvent

A polyphenolic root extract from Tagetes erecta, rich with ALB, is obtained according to the following process:
culturing Tagetes erecta in aeroponics with a defined nutrient solution, with a N/P/K composition corresponding to 15/10/30 and at an electroconductivity between 0.4 and 1.6 mS/cm,
stimulating the plant for a duration of 2 to 4 weeks by a nitrogen stress by the use of a N/P/K nutrient solution comprising: less than 6% of nitrogen, 15% of phosphorus and 40% of potassium, and an electroconductivity of 0.6 to 0.8 mS/cm,
extracting the molecules of interest by dipping the roots still bound to the plant for a duration of 15 to 30 minutes in a propane 1,2 diol/water mixture, at a ratio between 85/15 and 70/30, at room temperature and a pH of 3,
extracting the molecules of interest by macerating the plant cut off roots for a duration of 48 to 72 hours in a propane 1,2 diol/water mixture at a ratio between 85/15 and 70/30, at room temperature and a pH of 3,
mixing the extracts obtained in the two previous extraction steps,
the extract is purified (salt removal by nanofiltration), concentrated by nanofiltration and clarified by clarifying filtration, and
readjusting the propane 1,2 diol content of the extract (at least 50%) as well as the pH (around 3.5) of the extract for preserving the same.

The root extract of Tagetes erecta thus obtained has the following characteristics:
dry solid content: 13.3 g/L
estimated ALB1 content: 154 mg/L (that is 1.15% of the dry extract or 0.6% of the dry root biomass)
ALB2 and ALB3 content deduced: 813 mg/L (that is 6.1% of the dry extract or 3% of the dry root biomass)
total ALB content deduced (all the isomers): 967 mg/L (that is 7.3% of the dry extract or 3.6% of the dry root biomass)
solvent: 62%
pH: 3.66

ALB quantification is made thanks to a control series prepared from a commercial ALB1 standard provided by Extrasynthese (Genay, France) solubilised in the extraction solvent and acidified to pH 3.5 with phosphoric acid. The concentrations of all ALB isomers are thus expressed as ALB1 equivalents in each extract.

FIG. 1 shows a chromatogram of the root extract from Tagetes erecta obtained according to example 1. The apparatus used for analysing the extract is a UPLC Shimadzu Nexera X2 (LC-30AD pumps, SIL-30AC auto-sampler, CTO-20A furnace, SPD-M20A diode array detectors; Kyoto, Japan) operating in reverse phase with a Kinetex biphenyl column (00F-4622-AN, Phenomenex, Torrance, Calif., USA) with dimensions 150 mm×2.1 mm, 2.6 μm. The mobile phase consists of a solvent A (Eau ultrapure Mili-Q, Merck Millipore+0.1% formic acid, Carlo Erba, Val-de-Reuil, France) and a solvent B (Acetonitrile, Sigma-Aldrich Chemie GmbH, Steinheim, Germany) the gradient of which has been programmed the following way: 5-20% B (0-3.5 min), 20-27.5% B (3.5-8 min), 27.5-90% B (8-8.1 min), 90% B (8.1-10 min), 90-5% B (10-10.1 min), 5% B (10.1-12 min). The analysis flow rate is 0.5 mL/min with a furnace temperature of 40° C. At the column outlet, a diode array detector records UV spectra between 220 and 370 nm. The apparatus is coupled to a mass spectrometer (Shimadzu LCMS-2020) operating with electrospray ionisation (4.5 kV) in positive and negative mode in a range of m/z between 100 and 1000. The software LabSolutions (version 5.60 SP2) is used to run the system. The extract to be analysed is filtered through a 0.2 μm filter before injection.

### 1.2 Extract Prepared from a Hydroethanolic Solvent

Tagetes erecta are cultured in aeroponics with a culture medium 15/10/30 (nitrogen/phosphorus/potassium) having an electroconductivity of 0.4 to 1.6 mS/cm, and then the roots are stimulated for a duration of 2 to 4 weeks by a nitrogen stress by the use of a N/P/K nutrient solution comprising: less than 6% of nitrogen, 15% of phosphorus and 40% of potassium, and an electroconductivity of 0.6 to 0.8 mS/cm. The roots are cut off and then dried for 48 h in a ventilated stove at a temperature between 40 and 50° C. 450 mg of dried roots are dry milled and then macerated for 3 hours under stirring at room temperature in 45 mL of a 70/30 hydroethanolic solution (ethanol/water—v/v). Then, the extract to be analysed is filtered through a filter of 0.2 μm before UPLC injection according to the material and methods described above in example 1.1.

ALB quantification is made according to the material and methods described above in example 1.1.

The root extract from Tagetes erecta thus obtained has the following characteristics:
no ALA identified
ALB1 content estimated: 0.5% of the dry root biomass or 2.5% of the dry extract
ALB2 and ALB3 content deduced: 2.2% of the dry root biomass or 11% of the dry extract
total ALB content deduced (all the isomers): 2.7% of the dry biomass or 13.5% of the dry extract.

### Example 2: Preparation of a Root Extract and an Extract of Leaves from Tagetes patula Cultured in Aeroponics without a Stimulation Step and Characterisation of the ALB Content of these Extracts

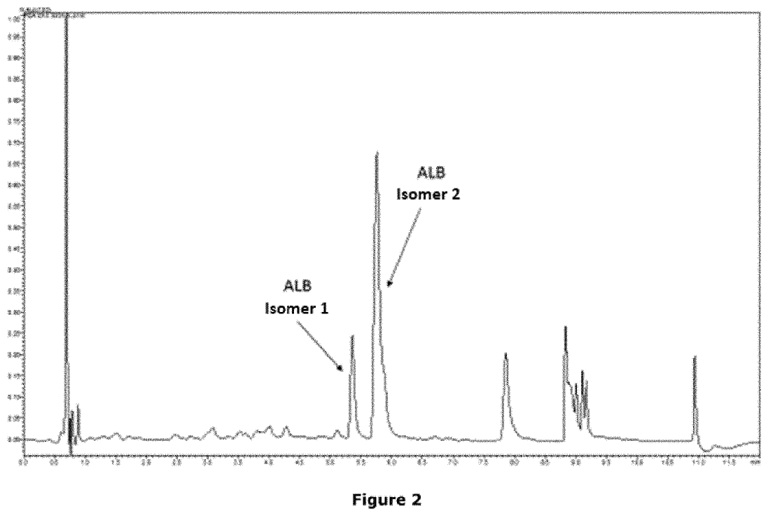

The extracts of roots or leaves are prepared from dried roots and leaves from Tagetes patula, originating from Reunion Island. The supplier is Societe Horticole de Bassin Plat, EARL Bassin Plat 31, Chemin de la Croix de Jubile 97410 Saint Pierre, Reunion Island. The 2-leaf stage plants are cultured in aeroponics with a culture medium 6/10/36 (nitrogen/phosphorus/potassium) having an electroconductivity of 0.7 mS/cm. After 2.5 weeks of culture in a aeroponic medium, fresh roots and leaves are cut off, and then dried for 48 h in a ventilated stove at a temperature between 40 and 50° C. 20 mg of dried tissues (leaves or roots) are dry milled and then macerated for 3 hours under stirring at room temperature in 2 mL of a 70/30 hydroethanolic solution (ethanol/water—v/v). Then, the extract is filtered through a 0.2 μm filter before UPLC injection according to the material and methods used for example 1. The root extract from Tagetes patula according to the chromatogram shown in FIG. 2 has the following characteristics:
no ALA identified
ALB1 content estimated: 0.07% of the dry biomass or 0.35% of the dry extract
ALB2 content deduced: 0.27% of the dry biomass or 1.35% of the dry extract
total ALB content deduced (all the isomers): 0.34% of the dry biomass or 1.7% of the dry extract. The presence of ALA or ALB has not been identified in the extracts of leaves from Tagetes patula.

ALB quantification is made thanks to a control series prepared from a commercial ALB1 standard provided by Extrasynthese (Genay, France) solubilised in an extraction solvent and acidified at pH=3.5 with phosphoric acid. The concentrations of all the ALB isomers are thus expressed in ALB1 equivalent in each extract.

FIG. 2 shows a chromatogram of the root extract from Tagetes patula obtained according to example 2, the material and methods used for performing chromatography are identical to the materials and methods of the chromatography of example 1.1.

### Example 3: Characterisation of a Root Extract from Tagetes erecta Relative to a Cosmetic Benefit—Target Identification by Analysis of the Gene Expression Modifications by qRT-PCR on TaqMan Cards

The study consists in measuring the effects of the extract from Tagetes erecta by qRT-PCR on microfluidic TaqMan cards, on the one hand, on the expression of 94 genes involved in dermis biology, conjunctive tissue remodelling and ageing (“Dermal Benefits” card defined by StratiCELL) and, on the other hand, on the expression of 94 genes involved in key epidermal functions, such as the barrier function directly related with moisturising, antioxidant response, or even pigmentation by melanocytes (“Epidermal Benefits” card defined by StratiCELL).

The protocol consisted in adding the extract from Tagetes erecta in the culture medium of Normal Human Dermal Fibroblasts (NHDFs) in single layer and reconstituted melanised human epidermises (RHE/MEL/001), and, after 24 h, analysing the different RNA populations to identify the genes differentially expressed by qRT-PCR.

A prior cytotoxicity study enabled the working concentration of the extract from Tagetes erecta to be defined for the gene expression study.

TGF-β1 (Transforming Growth Factor-beta-1) and vitamin D3 (1α,25-dihydroxyvitamin D3), the effects of which are documented in literature, have been used as reference molecules in order to validate the test systems and analysis method.

### 3.1. Materials and Methods

Extract

The Tagetes erecta plants are cultured in aeroponics according to example 1. The fresh roots are cut off and then macerated at room temperature for 24 hours in a 70/30 hydroethanolic solution (ethanol/water—v/v). Then, the extract is filtered. The solvent is removed by using a rotary evaporator and the powder is dried in a drier. A liquid phase chromatography analysis coupled with a mass spectrography (Agilent 1200 series) enabled the presence of leontopodic acid B to be checked in the extract.

Cell Culture

The first part of the study has been made on human dermis fibroblasts NHDF (ATCC, CRL-2522, origin: foreskin) cultured in single layer in a DMEM medium (Invitrogen, 31885-049) containing antibiotics (Penicillin/Streptomycin, Invitrogen, 15140-122) but containing no serum. These cells have been maintained in a wet atmosphere at 37° C. containing 5% of CO2.

The second part of the study has been made on reconstituted epidermises (StratiCELL®, RHE/MEL/001) containing or not containing primary human melanocytes NHEM (Normal Human Epidermal Melanocytes) from a dark phototype donor (phototype IV to V) (Invitrogen, C2025C, batch nº439684). The tissues have been cultured at the air-liquid interface for 14 days in an appropriate culture medium, and a wet atmosphere at 37° C. containing 5% of CO2.

Determining the Concentration Range of the Tagetes erecta Extract Study by a Preliminary Cytotoxicity Study

In order to determine the optimum analysis concentration for the Tagetes erecta extract, a preliminary experiment has been made on fibroblasts NHDFs, on human melanocytes NHEMs and on reconstituted epidermises.

The fibroblasts NHDFs have made 30.1 and 30.7 population doublings and the human melanocytes NHEMs have been seeded in 24-well plates 24 h before applying the active agents.

The reconstituted epidermises (RHE/001; batch CB0314/2) have been transferred in a 12-well plate before being treated with the extract.

The extract has been diluted in a culture medium, and then added, without a prior filtration, in the culture medium of fibroblasts NHDFs containing no serum, in the medium of the primary human melanocytes NHEMs, or in the culture medium of the differentiated epidermises.

This study consisted in evaluating the viability of cells and epidermises to MTS (3-(4,5-dimethythiazol-2-yl)-5-(3-carboxy-methoxyphenyl)-2-(4-sulphophenyl)-2H-tetrazolium) (Promega, G3581), 24 hours after adding the Tagetes erecta extract at 5 concentrations and 3 repeats (n=3) for the fibroblasts NHDFs and the melanocytes NHEMs, and at 2 concentrations and 3 repeats (n=3) for the reconstituted epidermises. The 2 concentrations tested for the reconstituted epidermises have been chosen based on the cytotoxicity results obtained for the melanocytes NHEMs.

SDS (sodium dodecyl sulphate) is toxic for the cells and has been used as a positive control in order to validate the experiment.

At the end of this experiment, a non-cytotoxic concentration has been defined in order to conduct the measurement of the expression modifications of the target genes. The following concentrations, in μg dry extract/mL, have been tested:
fibroblasts NHDFs and melanocytes NHEMs: 0.16 μg/mL, 0.8 μg/mL, 4 μg/mL, 20 μg/mL, 100 μg/mL;
reconstituted epidermises: 4 μg/mL and 20 μg/mL.

Analysis of the Gene Expression Modifications

The extract has been added, at a chosen concentration, in the culture medium of the human fibroblasts NHDFs (that made 30.5 and 30.8 population doublings) in the absence of serum or in the culture medium of the melanised differentiated epidermises (RHE/MEL/001, batches CB0314/3 and CB0314/4), without a filtration prior to cell/epidermis contacting.

Reference molecules have been studied in parallel, namely, TGF-β1 for fibroblasts NHDFs and vitamin D3 (VD3) for reconstituted epidermises.

Extraction of Total RNA

The extraction of total RNAs has been made using the RNeasy Mini kit (Qiagen, 74106). 24 h after adding the extract, the cells have been rinsed with PBS and lysed in the ad hoc lysis buffer, whereas the epidermises have been directly soaked in this buffer (culture triplicates have been made for each condition). The extraction and purification of RNAs have been made according to the supplier's instructions. The total RNAs have then been preserved at −80° C.

RNA Qualification by Spectrophotometry and Capillary Electrophoresis

The concentration of total RNAs has been determined by spectrophotometric measurement. The quality and integrity of RNAs have then been checked by capillary electrophoresis (Agilent Bioanalyzer 2100 platform—Agilent RNA 6000Nano Kit, 5067-1511).
Quantification of the RNAs by spectrophotometric measurement: an aliquot of each RNA has been diluted in RNAse-free water and its concentration has been determined using a Ultrospec 1100 Pro spectrophotometer (Amersham).
Integrity of the RNAs by capillary electrophoresis on Agilent Bioanalyser: the integrity of the total RNA has been evaluated by viewing the electrophoresis peaks corresponding to the ribosomal RNAs. For the total RNAs of upper eukaryotes, the size of the ribosomal bands should be 1.9 kb for 18S-RNA and 4.7 kb for 28S-RNA. The intensity of the band corresponding to 28S-RNA should be higher than the intensity of the band corresponding to 18S-RNA. Small diffuse bands representing RNAs with a lower molecular weight (RNAt and ribosomal RNA 5S) can be present. When RNA is degraded, a spread of the bands of ribosomal RNA as well as a background noise of the RNAs with a higher molecular weight are observed.

Synthesis of the Complementary DNAs or cDNAs

Reverse transcriptions (RT) have been made using the “High Capacity RNA-to-cDNA Kit” (Applied Biosystems, 4387406). For the synthesis of cDNAs, a mix has been prepared according to the supplier instructions, with 2 μg of total RNA, the ad hoc buffer provided in the kit and the reverse transcriptase enzyme. This reaction has been made at 37° C. for 1 hour, and then 5 minutes at 95° C. and finally the cDNA samples are put on ice and stored at −20° C.

Validation of the Test Systems—Real Time qPCR Using TaqMan Type Fluorescent Probes

The real time qPCR method has been used to quantify the expression of different specific targets in populations of ARNs from fibroblasts NHDFs treated with TGF-β1 (20 ng/mL) as well as melanised epidermises treated with vitamin D3 (100 nM) and by the solvent ethanol (0.1% EtOH), used to solubilise VD3.

The target sequences of the genes of interest have been amplified by PCR using the “TaqMan Gene Expression Assays” (Applied Biosystems). These kits comprise a TaqMan probe and 2 specific primers, which have been pre-mixed at a concentration of 18 μM for each primer and 5 μM for the probe. This mixture is concentrated 20 times. The TaqMan probes have been grafted with a fluorophore (FAM) at the 5′ end of the sequence and with a fluorescence “quencher” at the 3′ end.

The PCRs have been made using the 7900HT Fast Real-Time PCR system (Applied Biosystems). The reactions have been made in a 20 μL volume. The reaction mixture contains 10 μL TaqMan Fast Universal Master Mix (Applied Biosystems), 1 μL TaqMan Gene Expression Assay and 5 μL RNase-free water.

In each well of a 96-well microplate, 16 μL of the mixture and 4 μL of cDNA (4 ng) have been added. For normalisation purposes, reaction mixtures with probes and primers corresponding to glyceraldehyde-3-phosphate dehydrogenase (GAPDH) for the fibroblasts and β2-microglobulin (B2M) for the melanised epidermises have also been prepared with the same cDNA samples. A cDNA-free control was used as an amplification negative control. The thermal cycles have been programmed with an incubation step at 50° C. for 2 min, followed by a first denaturation step at 95° C. for 10 min. The PCR amplification protocol was continued with 40 cycles of 15 sec at 95° C. followed by one min at 60° C. The expression levels have been quantified according to the method of calculating the relative expression with respect to a housekeeping gene (2^{-ΔCt}), derived from calculation of ΔΔCts (Pfaffl, A new mathematical model for relative quantification in real-time RT-PCR, Nucleic Acids Res, 29 (9), 2001, 2002-2007; Livak and Schmittgen, Analysis of relative gene expression data using Real-Time Quantitative PCR and the 2^{-ΔCt} method, Methods, 25 (4), 2001, 402-408) described hereinafter.

The relative quantification of the transcripts has been made by using a calculation method which consists in comparing the average of the control values (Ct) obtained for the conditions TGF-β1 (20 ng/mL) and VD3 (100 nM) with the respective control conditions (non-treated CTL and 0.1% ethanol CTL). These Cts represent the detection threshold from which the amount of DNA is such that the signal is significantly distinguished from the background noise. This Ct average value has been normalised with respect to the housekeeping gene, that is Glyceraldehyde-3-phosphate dehydrogenase (GAPDH) for fibroblasts NHDFs and β2-microglobulin (B2M) for reconstituted melanised epidermises. Thus, the difference in the expression level (RQ) has been obtained by using the formula:

RQ=2^{-(ΔCt treated condition-ΔCt reference condition)}

where ΔCt=Ct (target gene)−Ct (housekeeping gene) within a same cDNA sample.

Preparation of Taqman Microfluidic Cards, Performance of the Quantitative PCR and Analysis of the Cts

The reaction mixtures for PCR on TaqMan microfluidic cards, tailored-made by Applied Biosystems, have been prepared following the detailed instructions of the Applied Biosystems Micro Fluidic Card Getting Started Guide. In summary, 100 ng of cDNA have been added to a mixture specific to PCR (Taqman Universal PCR Master Mix, 4364338, Applied Biosystems) before being injected in the card and dispersed by capillarity. After the card has been centrifuged, the same has been sealed before the performance of the quantitative PCR and analysis with the 7900HT system from Applied Biosystems, using the software ABI PRISM® 7900 Sequence Detection System, SDS2.4.

The threshold cycles (Ct) have been obtained for all the genes represented in the cards and expressed by the fibroblasts NHDFs and the melanised reconstructed epidermises.

The results have been exported from the real time qPCR device using the software SDS RQ Manager (v1.2.1, Applied Biosystems) and the analysis of the expression modifications has been made using the software Data Assist (V3.0., Applied Biosystems) designed to make the relative quantification of the gene expression using the method of comparison of the Cts (AACts) (Pfaffl, 2001 and Livak and Shmittgen, 2001) described in the paragraph above and a combination of statistical analyses. As set out above, the ΔΔCt calculation method consists in comparing Ct values obtained for the conditions treated by the extract with the reference condition (DMSO control). These Ct values have themselves been normalised with respect to the housekeeping gene GAPDH (glyceraldehyde-3-phosphate dehydrogenase) for the fibroblasts NHDFs and B2M (β-2 microglobulin) for the melanised reconstructed epidermises. The permissible maximum Ct value used as a detection threshold has been set to 36 cycles.

### 3.2. Results

### 3.2.1. Determination of the Extract Analysis Concentration by a Cytotoxicity Study

The prior cytotoxicity study on the fibroblasts NHDFs, the human melanocytes NHEMs and on the reconstituted epidermises enabled a working concentration (prepared in DMSO) to be defined for the gene expression study. The solvent present in the extract has been tested in parallel. 0.08% (for NHDFs) and 0.05% (for NHEMs) SDS has been used as a cytotoxicity positive control in order to validate the experiment. Based on these results (data not shown), the following concentrations have been chosen for the rest of the study: 100 μg of dry extract/mL (for the fibroblasts NHDFs) and 20 μg of dry extract/mL (for reconstituted melanised epidermises).

### 3.2.2. Qualification of the RNAs by Capillary Electrophoresis

The different RNA populations actually demonstrate the presence of narrow peaks, corresponding to the ribosomal RNAs 18S and 28S, and a balanced ratio between both peaks. The absence of intermediate and spread peaks, which are characteristic of RNA degradation products reflects the integrity of the different populations (data not shown). The quality and integrity of the extracted RNAs being demonstrated, consequently these have been used for continuing the protocol and have been engaged in the complementary DNA synthesis reactions.

### 3.2.3. Effects of the Extract on 94 Target Genes within the Fibroblasts NHDFs

The extract has been added in the culture medium of the fibroblasts NHDFs (n=3).

Controls only treated by the solvent 1% DMSO (vehicle of the extract) have also been analysed. In parallel, TGF-β1 (20 ng/mL) has been studied as a validation control. After 24 hours of culturing the fibroblasts NHDFs, the total RNA populations have been extracted, their integrity has been analysed by capillary electrophoresis, and the gene expression differences have been analysed by qRT-PCR using 96-well TaqMan cards, targeting the key dermis functions.

The results are shown as a summary in the form of a table (Table 2) indicating genes representative of a beneficial cosmetic effect, varying significantly, 24 hours after applying the extract on human dermis fibroblasts NHDFs.

TABLE 2
List of genes that significantly vary 24 hours after
applying the root extract from Tagetes erecta (100
μg ES/mL) on human dermis fibroblasts NHDFs.

| | | Extract | |
|---|---|---|---|
| Symbol no test | Name of the gene | RQ | p-value |
| SOD2- | Superoxide dismutase 2, | 13.58 | 0 |
| Hs00167309_m1 | mitochondrial | | |
| CCL5- | Chemokine (C-C motif) | 2.24 | 0.0368 |
| Hs00982282_m1 | ligand 5 | | |
| HMOX1- | Heme oxygenase | 2.05 | 0.0197 |
| Hs01110250_m1 | (decycling) 1 | | |
| DCN- | Decorin (PGS2) | 1.77 | 0.0396 |
| Hs00754870_s1 | | | |
| SOX2- | Transcription | 1.71 | 0.0361 |
| Hs01053049_s1 | factor SOX-2 | | |
| HPSE- | Heparanase | 1.66 | 0.0017 |
| Hs00935036_m1 | | | |
| TXNRD1- | Thioredoxin | 1.56 | 0.0199 |
| Hs00917067_m1 | reductase 1 | | |
| NQO1- | NAD(H) dehydrogenase, | 1.51 | 0.0025 |
| Hs02512143_s1 | quinone 1 | | |
| MT2A- | Metallothionein 2A | 1.51 | 0.0208 |
| Hs02379661_g1 | | | |
| COL3A1- | Collagen 3 alpha 1 | 1.43 | 0.002 |
| Hs00943809_m1 | subunit (COL3A1) | | |
| CYR61- | Protein CYR61 | 1.43 | 0.0293 |
| Hs00998500_g1 | | | |
| COL16A1- | Collagen subunit 16 | 1.42 | 0.034 |
| Hs00156876_m1 | alpha 1 (COL16A1) | | |
| NGF- | “Beta-nerve | 1.32 | 0.0036 |
| Hs00171458_m1 | growth factor” | | |
| FBLN5- | Fibulin-5 | 1.29 | 0.0062 |
| Hs00197064_m1 | | | |
| MYC- | Myc proto- | 1.26 | 0.039 |
| Hs00153408_m1 | oncogene protein | | |
| G6PD- | Glucose-6-phosphate | 1.18 | 0.0411 |
| Hs00166169_m1 | dehydrogenase | | |
| NTRK2- | BDNF/NT-3 receptor | 0.56 | 0.0078 |
| Hs00178811_m1 | growth factors | | |
| MKI67- | Antigen Ki-67 | 0.48 | 0.0111 |
| Hs01032443_m1 | | | |
| NGFR- | Member 16 of the tumour | 0.06 | 0.0006 |
| Hs00609977_m1 | necrosis factor receptors | | |
| | superfamily (p75NTR) | | |

The symbol of genes and test number, name of genes, relative expression (RQ) with respect to the vehicle 1% DMSO (RQ > 1: increase - RQ < 1: decrease) and the p-value are shown.

Antioxidant Defence (6 Overexpressed Genes)

The extract from Tagetes erecta sharpely induces the gene expression of superoxide dismutase 2 (SOD2) (X 13.6). This regulation is coupled with the overexpression of other genes involved in the response to oxidative stress, such as HMOX1 (heme oxygenase 1 (X2,1)), TXRND1 (thioredoxin reductase 1 (X1,6)), NQO1 (NAD(H) dehydrogenase, quinone 1 (X1,6)), MT2A (metallothionein 2A (X1,5)) and G6PD (Glucose-6-phosphate deshydrogenase (X 1,2)).

Superoxide dismutase 2 coded by the gene SOD2 is a mitochondrial protein involved in the front line defence against reactive oxygen species. Indeed, it converts the superoxide anion into hydroperoxide which in turn, is converted into oxygen and water by catalase and peroxiredoxines.

The NAD(H) dehydrogenase, quinone 1 (NQO1) is another detoxifying enzyme which promotes, by reduction, the formation of hydroquinones from quinones, preventing radical species for being produced. The gene NQO1 is overexpressed in response to pro-oxidant agents, to heavy metals, to UVs or even to ionising radiations in order to protect the cell from their deleterious effects.

Glucose-6-phosphate dehydrogenase coded by the gene G6PD is the first enzyme of the pentose phosphate pathway. It catalyses the oxidation of glucose-6-phosphate into 6-phosphoglucono-5-lactone. This reaction is coupled with the reduction of a NADP+ molecule into NADPH, necessary for reduction of glutathion GSSG (oxidised form) into glutathion GSH (reduced form), a powerful antioxidant.

Metallothioneins, including metallothionein 2A (MT2A), are low molecular weight proteins capable of binding metals. They are involved in various biological functions such as free radical neutralisation, zinc storage or even protection against cadmium toxicity.

The gene HMOX1 coding for the protein HO-1 or heme oxygenase is often overexpressed in response to a stress, and plays a crucial role in protecting and maintaining cellular homeostasis. The protein HO-1 is involved in heme degradation (having pro-oxidant properties) into carbon monoxide, ferrous iron and biliverdin. The latter 2 components are the precursors of the antioxidants bilirubin and ferritin. HMOX1 is thus known for its protective role against oxidative stress.

The thioredoxin system is one of the major antioxidant defence systems. In this system, thioredoxin reductase catalyses transfer of electrons from NADPH (Nicotinamide Adenine Dinucleotide Phosphate) to thioredoxin, which in turn exerts a reductive action on various target proteins. Among the 3 thioredoxin reductase isoforms, the isoform 1 coded by the gene TXRND1 is the most studied. It is known to be in particular under the control of the transcriptional control of the transcription factor Nrf-2 playing a major role in the anti-oxidant defence.

The overexpression of 6 genes involved in the anti-oxidant defence reflects the capacity of the extract to reduce the effects of pro-oxidant stresses generating an excess of free radicals, as UVs accelerating age signs at the skin, or even to fight against various pathologies or pro-inflammatory conditions, also generating reactive oxygen species.

The extract is clearly suitable in a cosmetic claim related to the control of free radical production, for anti-age or anti-inflammatory purposes.

Skin Resistance and Elasticity

Several genes coding for the extracellular matrix (MEC) proteins are also induced: DCN, HPSE, COL3A1, COL16A1, FBLN5.

Decorin (DCN) intervenes, at the dermis, in the collagen fibril assembly. Collagens III (COL3A1) and XVI (COL16A1) play a role in maintaining the MEC homeostasis and are thus important for the dermis resistance and elasticity. Heparanase encoded by the gene HPSE plays a role in the degradation of heparan sulphates (HS), which are components of the basement membrane present at the cell membranes where they are associated with other proteins to form proteoglycans (among others perlecan, syndecans and glypicans). Fibulin-5 (FBLN5) plays in turn a major role in assembling the elastic fibres at the dermis.

These regulations thus position the extract as being potentially interesting in order to promote dermis restructuration within the context of skin ageing.

Neurotrophins and Pain Perception

Several genes are also repressed by the extract: such as the case of NTRK2 (X0,6) and NGFR (X0,06). The latter code for neurotrophin receptors, responsible for pain sensation. The under expression of these genes could promote a soothing or calming effect on skin due to the inflammatory condition or an irritation sensation which develops after shaving, friction, or contact with allergens.

Neurotrophins are part of a growth factor family controlling development, maintenance and apoptosis of neuronal cells. Neurotrophins also exert multiple non-neuronal functions: they regulate cellular proliferation and differentiation, tissue apoptosis and remodelling in particular at skin.

Dermis fibroblasts as well as myofibroblasts synthesise and secrete neurotrophins such as Nerve Growth Factor (NGF), Brain-derived neurotrophic factor (BDNF) or even neurotrophin-3 (NT-3). The effects of neurotrophins are mediated by their receptors also expressed by fibroblasts. Two class of receptors are distinguished: receptors of the tyrosine kinase receptors (Trk) family and the neurotrophin receptor p75 (p75 NTR or NGFR). Each receptor has an affinity and a specificity unique to its ligand(s). TrkB (coded by the gene NTRK2) specifically binds BDNF as well as NT-3 and NT-4. P75 NTR is in turn capable of binding all the neurotrophins.

The neurotrophins, via their receptors, stimulate fibroblast proliferation and migration as well as differentiation of fibroblasts into myofibroblasts. Therefore, they could be involved in tissue remodelling regulation during the healing process of skin wounds.

NGF is a neurotrophic factor, initially described as a neuron survival factor in the central nervous system, but also at the skin sensorial neurons or nociceptors. NGF also plays a role in inflammation. Indeed, NGF neutralisation inhibits the sensitisation of sensory neurons (nociceptors), resulting in reducing pain perception associated with an inflammatory state.

The extract induces a decrease in the expression of genes coding for neurotrophin receptors, including in particular receptors TrkB (NTRK2) (X 0.06) and p75 NTR (NGFR) (X 0.06) and NGF. This could thus promote a skin soothing or calming effect due to an inflammatory condition or an irritation feeling which is developed after shaving, friction or contact with allergens.

The extract promotes a skin soothing or calming effect due to an inflammatory condition or an irritation feeling which is developed after shaving, friction, or contact with les allergens.

### 3.2.4. Effects of the Root Extract from Tagetes erecta on Target Genes within the Melanised Reconstructed Epidermises

The extract has been added in the culture medium of the reconstructed melanised epidermises (n=3). Controls treated by the solvent DMSO (0.2% for the reconstructed epidermises) in which the extract has been prepared have also been analysed. In parallel, vitamin D3 (100 nM) has been studied as a validation control. Since vitamin D3 has been solubilised in ethanol, a “ethanol solvent” condition has been used as a control condition. After 24 h of culture, the total RNA populations have been extracted, their integrity has been analysed by capillary electrophoresis, and the gene expression differences have been analysed by qRT-PCR using 96-well TaqMan cards, targeting the key epidermal functions.

The results are shown as a summary in a table (Table 3) indicating the genes representative of a beneficial cosmetic effect, varying significantly, 24 hours after applying the extract on epidermises.

TABLE 3
List of genes which significantly vary 24 hours after
applying the root extract from Tagetes erecta (at
20 μg ES/mL) on reconstructed epidermises.

Extract

| Symbol no test | Name of the gene | RQ | p-value |
|---|---|---|---|
| HRNR- | Hornerin | 3.6 | 0.0003 |
| Hs02340614_m1 | | | |
| MMP1- | Matrix Metalloproteinase-1 | 2.1 | 0.0076 |
| Hs00899658_m1 | | | |
| AQP5- | Aquaporin-5 | 1.9 | 0.0366 |
| Hs00387048_m1 | | | |
| LAMA3- | Laminin subunit | 0.9 | 0.0312 |
| Hs00165042_m1 | alpha-3 | | |
| HMOX1- | Heme oxygenase | 0.8 | 0.0384 |
| Hs00157965_m1 | (Decycling) 1 | | |
| LCE2B- | “Late cornified | 0.8 | 0.0373 |
| Hs00863535_g1 | envelope” protein 2B | | |
| LOR- | Loricrin | 0.8 | 0.0258 |
| Hs01894962_s1 | | | |
| RNASE7- | Ribonuclease 7 | 0.8 | 0.0061 |
| Hs00922963_s1 | | | |
| PLG- | Plasminogen | 0.7 | 0.0026 |
| Hs00264877_m1 | | | |
| LTF- | Lactotransferrin | 0.7 | 0.0025 |
| Hs00914334_m1 | | | |

The symbol of the genes and the test number, the name of the genes, the relative expression (RQ) with respect to the vehicle 0.2% DMSO (RQ > 1: increase - RQ < 1: decrease) and the p-value are shown.

Epidermal Barrier Function

The extract increases the expression of the genes HRNR and AQP5. Hornerin (HRNR) and aquaporin-5 (AQP5) both participate in maintaining the epidermal barrier function and moisturising.

The extract acts as an inducer for the expression of the gene coding for hornerin (HRNR), with an amplitude of 3.6. Hornerin is a protein present at the horny layer and the granular layer of the epidermis. Recent works demonstrate that this protein is structurally close enough to pro-filaggrin (filaggrin precursor).

Hornerin is a protein essential to the mechanical strength of the horny layer. Indeed, it is a major component of the cornified envelop, the transglutaminase-3 substrate and located at the periphery of corneocytes. An antimicrobial defence role has also been evidenced for some hornerin-derived peptides. It has also been demonstrated that HRNR is underexpressed in vivo in many atopic dermatitis cases. The fact that hornerin is induced by a UV skin treatment or after “tape stripping” suggests a role for this protein at the barrier function integrity of skin after restoring the same following stresses brought about by sun exposure or mechanical friction in connection for example with shaving. The extract also induces the overexpression of AQP5 (X1.9) coding for aquaporin-5, a membrane protein acting as a water carrier through the plasma membrane and thus playing an important role in epidermis moisturising.

Because of its positive effect on the expression of a protein (hornerin) essential to the horny layer and the granular layer of epidermis, the extract demonstrates a positive role in the integrity and efficiency of the epidermal barrier function.

Moreover, by inducing the overexpression of a protein (aquaporin-5) involved in water transport through the plasma membrane, the extract plays a positive role in skin moisturising.

Skin Healing

The root extract from Tagetes erecta induces the expression of the gene of metalloproteinase-1 MMP-1 by a factor of 2.1. The MMPs are metalloproteinases capable of cleaving most of the MEC components and to modify, by proteolysis, a number of important molecules for skin healing. In particular, MMP1 plays a major role in keratinocyte re-epithelialisation, essential in the healing process for skin wounds. MMP1 facilitates MEC assembly, cell elongation and migration, actin cytoskeleton reorganisation, and induces the activation of the kinase ERK (extracellular signal-regulated kinase) necessary to the motility and invasion ability of epithelial cells. Further, it has been shown that the protein MMP1 is overexpressed at the epidermis in response to skin wounds.

The overexpression of the gene MMP-1 at the epidermis by the extract enables it to be positioned as an active potentially promoting skin healing, in particular at the wound closure. The extract turns out to be very interesting from this point of view because it induces an expression amplitude of this gene by a factor of 2.1.

### Example 4: Effect of Root Extracts from Tagetes erecta in Propane 1,2 Diol, on Human Fibroblasts and Keratinocytes—Study on “GeneChip Human Gene” DNA Chips

In order to analyse the possibility that an extract according to the invention has a beneficial action on skin, a DNA chip transcriptomic study has been made from human fibroblasts (NHDFs) and human keratinocytes (NHEKs) treated for 24 hours by a root extract from Tagetes erecta.

The expression variations of the genes have been contextualised and biologically interpreted through the proprietary database “StratiCELL Skin Knowledge database”.

A prior cytotoxicity study made in ad hoc cellular models, namely human fibroblasts (NHDFs) and human keratinocytes (NHEKs), enabled the optimum concentration of the extract used for the study to be determined.

### 4.1. Materials and Methods

Root Extract

A root extract from Tagetes erecta has been prepared according to example 1.1.

Cell Culture

The study has been made on Normal Human Dermal Fibroblasts (NHDFs) (ATCC/LGC promoChem, CRL-2522, origin: foreskin) at about 40% of their in vitro proliferative potential. These cells have been cultured as a single layer in DMEM medium (Invitrogen, 31885-049) without serum and containing antibiotics (penicillin/streptomycin, Invitrogen, 15140-122).

On the other hand, the study has also been made on Normal Human Epidermal Keratinocytes (NHEK); Lonza, 0019206, origin: foreskin) cultured as a single layer in an Epilife medium containing HKGS (Human Keratinocyte Growth Supplement) and gentamycin.

The cells have been maintained in a wet atmosphere at 37° C. containing 5% of CO2.

Determination of the Study Concentrations Range of the Root Extract from Tagetes erecta by a Preliminary Cytotoxicity Study

In order to determine the optimum analysis concentration of the extract, a preliminary experience has been made on fibroblasts NHDFs and keratinocytes NHEKs.

Fibroblasts NHDFs and keratinocytes NHEKs have been seeded in 24-well plates 24 hours before applying the extract, in a DMEM medium (INVITROGEN, 31885) containing antibiotics (penicillin/streptomycin, INVITROGEN, 15140) in the presence of 10% of serum for NHDFs or in an Epilife medium (INVITROGEN, M-EPI-500 A) containing the supplement HKGS (INVITROGEN, S-001-K) and gentamycin (INVITROGEN, 15710-049) for NHEKs.

The extract has been solubilised in the appropriate culture medium, and then placed in contact with the fibroblasts NHDFs or keratinocytes NHEKs for 24 hours. This study consisted in evaluating cell viability to MTS (3-(4,5-dimethythiazol-2-yl)-5-(3-carboxy-methoxyphenyl)-2-(4-sulphophenyl)-2H-tetrazolium) (Promega, G3581), 24 hours after adding the extract and its solvent (60% propane 1,2 diol pH=3.5) at 5 concentrations and 3 repeats (n=3).

SDS (sodium dodecyl sulphate) is toxic for cells and has been used as a positive control in order to validate the experiment.

At the end of this experiment, a non-cytotoxic concentration has been defined in order to conduct the transcriptomic analysis.

The following concentrations, in v/v percent, have been tested for the extract and solvent:

fibroblasts NHDFs and keratinocytes NHEKs: 3%, 1%, 0.33%, 0.11%, 0.037%.

Treatment of the Cells

The extract or solvent has been applied at a chosen concentration, for 24 h, in the culture medium of the fibroblasts NHDFs and keratinocytes NHEKs. Culture quadruplicates have been made for each condition (n=4).

Extraction of Total RNA

At the end of the treatments, the total RNA populations have been extracted. The extraction has been made using the RNeasy kit (Qiagen). After 24 h of treatment, the cells have been rinsed with PBS and lysed in ad hoc buffer. The RNA extraction and purification have been made according to the supplier's instructions. The total RNAs have then been preserved at −80° C. for the transcriptomic analysis.

Qualification of the RNAs by Spectrophotometry and Capillary Electrophoresis

The concentration of the total RNAs has been determined by spectrophotometric measurement. The RNA quality and integrity have then been checked by capillary electrophoresis (Agilent Bioanalyzer 2100 platform Agilent RNA 6000Nano Kit, 5067-1511).

Quantification of RNAs by spectrophotometric measurement: an aliquot of each RNA has been diluted in RNAse-free water and its concentration has been determined using a Ultrospec 1100 Pro spectrophotometer (Amersham).

Integrity of the RNAs by capillary electrophoresis on Agilent Bioanalyser: the integrity of the total RNA has been evaluated by viewing the electrophoresis peaks corresponding to the ribosomal RNAs. For total RNAs of upper eukaryotes, the size of the ribosomal bands should be 1.9 kb for 18S-RNA and 4.7 kb for 28S-RNA. The intensity of the band corresponding to 28S-RNA should be higher than the intensity of the band corresponding to 18S-RNA. Small diffuse bands representing lower molecular weight RNAs (RNAt and ribosomal RNA 5S) can be present. When RNA is degraded, a spread of the bands of the ribosomal RNA as well as a background noise of higher molecular weight RNAs are observed.

Hybridisation Phase on GeneChip Human Gene 2.0 ST Chips (Affymetrix)

The RNAs have then been diluted at 50 ng/μL and 50 μL of each sample (n=3) have been used. The fourth replica acts as a back-up.

The RNA samples (50 ng) have been amplified by the use of the Ribo-SPIA technology, according to 3 steps (Ovation Pico WTA System V2, NuGEN, 3302-12) and purified with the Agencourt RNA Clean up XP Beads kit (Agencourt—Beckam Coulter Genomics, A29168). For each sample, 5 μg have been fragmented and biotin-labelled, thanks to the NuGEN Encore Biotin Module (NuGEN, 4200-12). The hybridisation has been made on DNA chips from the GeneChip Human Gene 2.0 ST model (Affymetrix, 902112). The chip hybridisation, washing and binding steps have been made according to the protocol defined by Affymetrix. The hybridisation solution has been prepared by using the Affymetrix Genechip Expression 3′ Amplification Reagent Hybridization Controls (Affymetrix, 900454) and Hybridization Module for GeneChip Hybridization, Wash and Stain Kit (Affymetrix, 900720), and mixed with the complementary DNA (cDNA) amplified in the previous steps. The hybridisation has been made for 18 h in the GeneChip Hybridization Oven 640 (Affymetrix, 800139). Washing and development have been made using the GeneChip fluidics station 450 (Affymetrix, 00-0079) and the intensity measurements have been scanned with the GeneChip Scanner 3000 (Affymetrix).

Pre-Treatment of the Hybridisation Data

The treatment of the raw data has been made with the software R (v3.2.3) and package ‘oligo’ (v1.34.2) of the BioConductor project (v3.2; Gentleman R. et al., Genome Biology 2004, 5, R80). The latest version of bookshops provided by Affymetrix, constructed on the version 19 of the human genome (UCSC Human genome 19), and the RMA method, described by Irizarri et al. (Irizarry Ra. et al., Speed Tp. Biostatistics. 2003b, 4(2), 249-64; Irizarry Ra. et al., Boeke Jd. Stat Appl Genet Mol Biol. 2003a, 2, Epub. 2003 Mar. 18) have been used in order to guide and perform sequence pre-treatment and annotation.

Data Analysis, Annotation and Thematic Analysis

The individual statistical analysis of the genes/transcripts has been made with the “Moderated t” and “Moderated F” methods implemented in the package R Limma 3.26.8.

The gene annotation and gene group definition for the over-representation analysis have been made first based on the internal database “StratiCELL Skin Knowledge Database” (Salmon M & Berger F. Cosmetic expression 2014, 30, 91-94).

The over-representation analysis has been made with the hypergeometric test method for the purpose of characterising theme/group factors of dermo-cosmetic interest first from the proportion of their detected and differentially expressed targets. The analysis has been conducted first from the list of genes detected with a p-value of 0.05 and an expression level difference (variation rate or fold-change or FC) higher than 1.5 (bi-lateral).

### 4.2. Results

### 4.2.1. Determination of the Analysis Concentration of the Root Extract from Tagetes erecta by a Preliminary Cytotoxicity Study

A cytotoxicity study has been made on fibroblasts NHDFs and keratinocytes NHEKs. The extract and its solvent have been applied in culture media, during 24 h (n=3). The untreated control has been arbitrarily set to 100% of viability and the cytotoxicity threshold has been set at 80% of viability by convention. The SDS condition is the positive control which validates the experiment.

Based on the results from the preliminary cytotoxicity study (data not shown), the optimum concentrations (in v/v percent) selected for the extract and its solvent are the following ones: extract and solvent (propane 1,2 diol): for the fibroblasts NHDFs 3% and for the keratinocytes NHEKs 1%.

### 4.2.2. Quantification of the RNAs by Spectrophotometry

The absorbance ratio at 260 nm and 280 nm is used to evaluate RNA purity. A ratio close to 2 enables the sample to be considered as being pure and free of protein contamination. The ratio 260/230 is used as a secondary measurement for the sample purity. The value 260/230 is generally higher than the ratio 260/280 and is expected to be at about 2.2. The ratios obtained for all the samples of the present study are thus satisfactory (data not shown) and have then been qualified by capillary electrophoresis.

### 4.2.3. Qualification of the RNAs by Capillary Electrophoresis

The different RNA populations actually demonstrate the presence of narrow peaks, corresponding to the ribosomal RNAs 18S and 28S, and a balanced ratio between both peaks. The absence of intermediate and spread peaks, which are characteristic of RNA degradation products gives evidence of the integrity of the different populations (data not shown).

The quality and integrity of the extracted RNAs being demonstrated, therefore these have been used for continuing the protocol and been engaged in amplification, purification, biotin-labelling reactions and then hybridisation on DNA chips.

### 4.2.4. Analysis of the Gene Expression Modifications Induced by the Root Extract from Tagetes erecta

At the end of the transcriptomic study on human epidermis keratinocytes NHEKs, the root extract from Tagetes erecta could play a potential role in enhancing cohesion and epidermal differentiation, protection against environmental stresses and antimicrobial defence. However, the study on fibroblasts NDHFs revealed a few observations in connection with a detoxifying activity. Hereinafter, regulations observed in connection with these hypotheses are given in brackets, as follows: (gene name variation rate or fold change—p-value) and replaced within the context of skin biology functions modulated by the corresponding proteins.

### 4.2.4.1 Stimulation of the Epidermal Differentiation (Keratinocytes)

The human epidermis is a stratified epithelium making a barrier the layers of which are defined by the differentiation stage of the cells making it up. As they are differentiated, keratinocytes are filled with keratin filaments and contain a water-retaining matrix, the whole being surrounded by the cornified envelope. The keratinocytes in the differentiation terminal stage are isolated by intercorneocytic lipids and are attached to each other by corneodesmosomes.

Basement keratinocytes progress through the four epidermal layers: basement, spinous, granular and horny layers.

The basement layer is formed by a single seating of cubic or prismatic keratinocytes bound to each other through desmosomes. In this layer, the cells divide, one of both cells remains in the basement layer whereas the other migrates to the upper layers. Basement keratinocytes are attached through hemi-desmosomes to a basement membrane which separates the epidermis from the dermis and forms the dermo-epidermal junction.

The spinous layer is formed by 5 to 15 seatings of polygonal keratinocytes bounded to each other by desmosomes. These cells contain tonofilaments, which are keratin precursor filaments.

The granular layer is formed by 3 layers of keratinocytes which contain keratohyaline grains and lamelar granules.

The horny layer is formed by 5 to 15 layers of corneocytes, that is differentiated keratinocytes which have no longer nuclei and organites, but are surrounded by a cornified envelope. They are filled with keratin filaments.

Formation of Keratin Filaments

During the epidermal differentiation, keratin filaments (called tonofilaments) are increasing in number and begin to aggregate in the cells of the spinous layer to form tonofibrils. At the granular layer, they associate to keratohyaline granules. Keratohyaline granules are comprised of loricrin, involucrin (IVL−5.11−p=0,00000049), trichohyali and profilaggrine which will be transformed into filaggrin by different proteases, including caspase 14 (CASP14−10.63−p=0.0000014). Filaggrin cross-linking with keratin accelerates the aggregation and alignment process of tonofilaments enabling keratin cables which make up 85% of the corneocyte volume to be formed. They are then associated with desmosomes upon forming the cornified envelope by transglutaminases (TGM1−7.21−p=0,00000024). Keratin plays the role of a rigid mechanical structure in association with the cornified envelope to ensure maintenance of epidermal integrity.

At the basement layer, keratinocytes mainly express keratins 5, 14 and 15 (KRT15−1.9−p=0.00016) whereas the upper layers rather contain keratins 1 (KRT1−1.62−p=0.015), 2, 10 (KRT10−7.38−p=0.00000026) and 13 (KRT13−13.83−p=0,0000075).

Formation of the Cornified Envelope

During the keratinocyte differentiation stimulated by various transcription factors such as zinc finger protein 750 (ZNF750−9.42 p=0.00000065), the plasma membrane is gradually replaced with a rigid shell called a cornified envelope. The initiation of this change is reflected by an intercellular protein assembly coordinated by transglutaminases (TGM1−7.21−p=0,00000024). Periplakine (PPL−2.08−p=0,0002), envoplakin and involucrin (IVL−5.11−p=0,00000049) form a reinforcement which gradually covers the inner face of the plasma membrane. Simultaneously, the protein structure is reinforced by other proteins as loricrin, hornerin, “small prolin rich” proteins (SPRR1A−6.75 p=0,00000011; SPRR1B−5.71−p=0,0000007; SPRR2A−1.9−p=0.002; SPRR3−2.26−p=0,0007 and SPRR4−1.92 p=0.0039), proteins S100 (S100A7−2.46−p=0.0068; S100A8−6.55−p=0,00000016; S100A9−8.54−p=0,0000043; S100A12−2.43−p=0.0013), and “late-cornified envelope” proteins (LCE3D−1.99−p=0.0012; LCE1C−1.5−p=0.031).

Epidermal Barrier

To fully provide its functions, the epidermis requires maintaining a water gradient between the deep layers (water content ˜70%) and the surface layers (water content ˜20%). This gradient is maintained thanks to several systems, including mainly the intercorneocytic lipids, natural moisturising factor (NMF) and intercellular junctions.

The intercorneocytic lipids form a hydrophobic barrier mainly comprised of long-chain ceramides. Ceramide synthesis is ensured, among other things, by the acyl-CoA synthetase long chain family member 1 (ACSL1−2.08−p=0,0002) and the serine palmitoyltransferase long chain base subunit 3 (SPTLC3−1.79−p=0,00025). Ceramide transport is in turn made, among other things, by the “fatty acid binding protein 5 (FABP5−2.2 p=0,000036) and the ATP-binding cassette sub-family A member 12 (ABCA12−1.99−p=0.0017).

The Natural Moisturising Factor (NMF) is specific to the horny layer. It is comprised of 50% of amino acids and derivatives thereof obtained as a result of the proteolysis of filaggrin 16 by caspase 14 (CASP14−10.63−p=0,000014). Aquaporins, which are integral proteins of the plasma membrane, are another group of participants in aqueous transport and also play a crucial role in skin moisturising (AQP3−2.57−p=0,000019; AQP8−1.62−p=0.01).

Among the different types of intercellular junctions, the tight junctions are adhesion structures which control the paracellular passage of molecules, including water. They consist of several families of proteins: claudins (CLDN1−3.01−p=0,000035; CLDN7−1.6−p=0.0022) and occludin which enable plasmic membranes of two adjacent cells to move closer to each other. Desmosomes represent another type of junctions located between keratinocytes. Among the components of this type of structures, desmocollins (DSC1−8.24−p=0,00000059; DSC2−2.78−p=0,0000035; DSC3−1.61 p=0,00057) and desmogleins (DSG1−10.06 p=0,0000013; DSC3−1.98 p=0,000048) are transmembrane glycoproteins belonging to the cadherin family. Their extracellular parts ensure intercellular adhesion whereas their cytoplasmic parts are associated with intracellular proteins such as plakoglobin (JUP−1.93−p=0,00082), plakophilin (PKP1−1.99−p=0,00019) or even desmoplakin (DSP−1.78 p=0,00017). Desmoplakin interacts itself with keratin filaments thus enabling the cytoskeleton to be attached to the adhesion complex. During cornification, desmosomes are modified, the desmosomal plaque is incorporated to the cornified envelope and corneodesmosin is integrated therein. Thereby, they are called corneodesmosomes.

Grainyhead-like transcription factors (GRHL1−3.24−p=0,000031; GRHL3−6.28−p=0,0000037) also play an essential role in the formation of the epidermal barrier and its regeneration after an injury. GRHL3 is involved in the pathway controlling the keratinocyte polarity within the epidermis, an essential pathway to ensure an organised morphology of tissue and its repair.

### 4.2.4.2 Protection Against Environmental Stress (Keratinocytes and Fibroblasts)

Skin is a barrier against environmental stresses. To cope with these stresses that are pollutants and xenobiotics, keratinocytes and fibroblasts overexpress a set of genes capable of neutralising them and maintaining cellular balance.
Cytochromic proteins P450 of keratinocytes (CYP1A1−7.36−p=0,000047; CYP1B1−5.87−p=0,0000039; CYP4B1−1.52−p=0.013) and fibroblasts (CYP1B1−2.99−p=0,000012) are hemoproteins which intervene in oxydo-reduction reactions of metabolites or xenobiotics (monooxygenases). CYPs are highly involved in biodegradation of many exogenous molecules and participate in detoxifying the organism.

Aldehyde dehydrogenase 3A1 catalyses the transformation of aldehydes into acids. For keratinocytes, the gene expression is increased (ALDH3A1 −1.53 p=0.025). This dehydrogenase is involved in detoxifying the alcohol-derived acetaldehyde, in the corticoid metabolism and lipid peroxidation.
The expression of genes coding for heat shock proteins (HSPs) is increased by keratinocytes (HSPA4L−1.57−p=0.0021; HSPB1−2.77−p=0,000011; HSPB3−2.88−p=0.0011). HSPs are chaperone proteins having the main function of managing a response to stresses (infection, inflammation, exercise, toxin exposure, famine, hypoxia, water deficiency, etc.). Crystallin alpha B the expression of which is also increased for keratinocytes (CRYAB−1.96−p=0,00012) also plays the chaperone role in case of stress to prevent proteins from being aggregated. These chaperone proteins can be aided by co-chaperone proteins, as for example BCL2-associated athanogene the gene expression of which is increased for keratinocytes (BAG1−1.66 p=0,00053).
The cell has available toxicity regulators such as metallothioneins the expression of two genes of which is increased for fibroblasts (MT1G−11.19 p=0,00000079 and MT1H−3.91−p=0,00023), small proteins which are characterised by their high affinity to metal ions (arsenic, cadmium, etc.). They are directly associated to homeostasis regulation of essential cations and the cell oxydo-reduction potential.

Ferritin the FTL gene of which is increased for keratinocytes (FTL light chain−1.58−p=0.0023) is in turn responsible for iron detoxification.
To cope with detrimental UV radiation effects, and more particularly reactive oxygen species (ROS), the organisms have developed a sophisticated defence system which can be in some cases unbalanced or “overwhelmed”, that is called the oxidative stress. From the battery of proteins in charge of coping with this oxidative stress, there are NADPH deshydrogenase quinone 1 the gene expression of which is increased for keratinocytes (NQO1−1.65−p=0,00063) and heme oxygenase 1 the gene expression of which is increased for fibroblasts (HMOX1−3.01−p=0,000013). The redox potential of the cell cytoplasm is maintained thanks to glutathion, a tripeptide partly synthesised by glutamate cystein ligase the gene GCLC expression of which is increased for keratinocytes (GCLC−2.07−p=0,000065), from L-cystein and L-glutamate. Glutathion enables the organism to be detoxified from heavy metals but also ROSs to be removed since glutathion peroxidase the gene GPX2 expression of which is increased for keratinocytes (GPX2−1.8−p=0,00038) uses glutathion to reduce peroxides via an oxydo-reduction reaction.

### 4.2.4.3 Stimulation of the Anti-Bacterial Defence (Keratinocytes)

To fight against pathogenic aggressions, keratinocytes can produce antimicrobial peptides, responsible for the attraction and activation of immune cells.

Antimicrobial peptides have a wide spectrum activity and destroy organisms by disturbing their membrane integrity. Some of them are constitutively expressed but most of them are inducible. The main peptides are defensins (DEFB1−2.29−p=0.0027) and cathelicidin. Keratinocytes express other peptides of this type in particular calgranulin A (S100A8−6.55−p=0.00000016) and calgranulin B (S100A9−8.54−p=0.0000043) which form an antimicrobial heterodimeric complex called calprotectin. It has been demonstrated that calprotectin exerts its antibacterial activity against Escherichia coli, Klebsiella spp., Staphylococcus aureus, Staphylococcus Epidermidis, Capnocytophaga sputigena and Borrelia burgdorferi (Lyme disease). Calgranulin C (S100A12−2.43−p=0.0013) in turn sequestrates zinc and copper necessary for the growth of some bacteria (Helicobacter pylori) and deprives them of their nutriments.

On the other hand, the transcriptomic study enabled a very significant increase in the expression of the gene coding for chemokine (C—X—C motif) ligand 14 (CXCL14−6.4−p=0,00000035) to be evidenced. This chemokine is constantly produced in the epidermis and dermis under healthy conditions. However, in addition to its chemo-attractant effects, it has a recognised bactericidal activity against Escherichia coli, Staphylococcus aureus, Finegoldia magna, Streptococcus pyogenes.

Different mechanisms are developed to modulate bacteria proliferation, among those, the desquamation process which enables undesirable micro-organisms set on corneocytes to be removed is to be distinguished. Desquamation relies on the balance between the synthesis of structural proteins of corneodesmosomes and their degradation by specific proteases as kallikreins (KLK5−2.82−p=0,0000066; KLK7−6.47−p=0,00000012) and cathepsin L2 (CTSV−2.73−p=0,0000062). This balance is also controlled by protease inhibitors, in particular the secretory leukocyte peptidase inhibitor (SLPI−5.95−p=0,0000012). In addition to this function, SLPI is recognised as being an antimicrobial since the 1980's.

### 4.3. Conclusion

By positively modulating the genes coding for proteins involved in the epidermal differentiation, the enhancement of the cornified envelope, cellular junctions, moisturising, ceramide synthesis and transport, the root extract from Tagetes erecta could potentially stimulate epidermal turnover. The extract could also enhance epidermis cohesion and its barrier functions, limit water losses and promote tissue moisturising in this way.

The application of the extract from Tagetes erecta on cellular cultures of keratinocytes and fibroblasts suggest on the other hand a potentially protective activity to environmental stresses following induction of the expression of the genes coding for metallothioneins, for enzymes involved in the xenobiotic metabolism and for proteins and enzymes responsible for the antioxidant defence.

Moreover, it could also play a role in the epidermal antimicrobial defence by increasing the expression of the genes coding for some antimicrobial peptides or for chemokines involved in antibacterial and antifungal defence.

### Example 5: Effect of a Root Extract from Tagetes erecta on the Survival of Primary Cortical Neurons Injured by Aβ Peptide (20 μg, 24 Hours) and on a Neuritic Network of Primary Cortical Neurons Injured by Aβ Peptide (20 μg, 24 Hours)

The purpose of this study is to evaluate the effects of the root extract on rat primary cortical neurons, intoxicated by Aβ peptide according to the in vitro Alzheimer disease model described in Callizot et al., 2013 (Callizot N. et al., Operational dissection of β-amyloid cytopathic effects on cultured neurons, J Neurosci Res. 2013, 91: 706-16). The neuron survival as well as the integrity of the neuritic network are evaluated.

### 5.1. Materials and Methods

Root Extract

The root extract is prepared from dried roots from Tagetes erecta. The plants are cultured in aeroponics according to example 1. The fresh roots are cut off and dried. 70 g of dried roots are dry milled and then macerated for 65 hours in 1 litre of methanol. Then, the extract is filtered. The solvent is removed by using a rotary evaporator and the powder is dried with a drier to finally obtain 4.15 g of powder. The chromatographic analysis made according to example 1.1 enabled the presence of leontopodic acid B in the extract to be shown.

Four concentrations are tested: 10 ng/mL, 100 ng/mL, 1 μg/mL and 10 μg/mL of dry extract diluted in water with 0.1% of DMSO.

The vehicle used is the culture medium (see the section below) containing 0.1% of DMSO (Pan Biotech, Batch: H130813).

Cell Culture of Cortical Neurons

Rat cortical neurons are cultured as described in Singer et al., 1999 (Singer C, et al., Mitogen-activated protein kinase pathway mediates estrogen neuroprotection after glutamate toxicity in primary cortical neurons. J Neurosci. 1999, 19: 2455-2463) and Callizot et al., (2013). In summary, gravid female rats are killed by cervical dislocation after 15 days of pregnancy. The foetuses are collected and immediately placed on an ice L15 Leibovitz medium (Pan Biotech, Batch: 8810315) added with 2% of penicillin (10,000 U/mL) and a streptomycin solution (l0 mg/mL) (PS; Pan Biotech, batch: 1451013) and 1% of bovine serum albumin (BSA; Pan Biotech, batch: K180713). The cortex is treated for 20 min at 37° C. with a trypsin—EDTA solution (Pan Biotech, Batch: 3330914) at a final concentration of 0.05% of trypsin and 0.02% of EDTA. The dissociation is stopped by adding DMEM medium (Dulbecco's modified Eagle's medium) with 4.5 g/L of glucose (Pan Biotech, Batch: 8530315), containing DNase I grade II (final concentration 0.5 mg/mL; Pan Biotech, Batch: H140508) and 10% of foetal calf serum (FCS; Invitrogen, Batch: 41Q1613K). The cells are mechanically dissociated by three forced passages through 10 mL pipette cones. The cells are then centrifuged at 515 g for 10 min at 4° C. The supernatant is discarded, and the pellet is suspended in a defined culture medium consisting of a Neurobasal medium (Invitrogen, batch: 1715722) with a solution of 2% of supplement B27 (Invitrogen, batch: 1709534), 2 mmol/L L-glutamine (Pan Biotech, batch: 6620314), 2% of PS solution, and 10 ng/mL of brain derived neurotrophic factor (BDNF; Pan Biotech, Batch: H140108). The viable cells are counted with a Neubauer cytometer, using the trypan blue exclusion test. The cells are seeded at a density of 30,000 per well of a 96-well plate pre-coated with poly-L-lysine (Biocoat, Batch: 21614030) and are cultured at 37° C. in an incubator containing 95% of air and 5% of CO2. The medium is changed every day. After 11 days of culture, the cortical neurons are intoxicated by a solution of Aβ1-42 peptides (see below).

Preparation of Aβ1-42 Peptide and Exposure of the Root Extract to Said Peptide

The preparation of Aβ1-42 peptide is made according to the procedure described in Callizot et al., (2013). In summary, the Aβ1-42 peptide (Bachem, Batch: 1014012) is dissolved in the defined serum-free culture medium mentioned above, at an initial concentration of 40 μmoles/L. This solution is gently stirred for 3 days at 37° C. in the dark and immediately used after being diluted in the culture medium at the concentration tested (20 μM).

On the eleventh day of culture, the root extract tested at 4 different concentrations is dissolved in the culture medium and is then pre-incubated with the primary cortical neurons for 1 hour before applying the Aβ1-42 peptide. The Aβ1-42 peptide solution is added at a final concentration of 20 μM diluted in the culture medium in the presence of the extract to be tested and the whole is left for 24 hours.

Evaluation of the Neuron Survival and the Integrity of the Neuritic Network

24 hours after intoxication by Aβ1-42 peptide, the cell culture supernatant has been removed and the cortical neuron cultures have been fixed by a cold solution of ethanol (95%, Sigma, Batch: SZBD3080V) and acetic acid (5%, Sigma, Batch: SZBD1760V) for 5 min at −20° C. After permeabilisation with 0.1% of saponin (Sigma, Batch: BCBJ8417V), the cells are incubated for 2 hours at room temperature, with an anti-microtubule-associated-protein 2 mouse antibody (MAP-2, Callizot et al., A new long term in vitro model of myelination. Experimental Cell Research, October 2011. Volume 317, Issue 16, Pages 2374-2383), Sigma, batch: 063M4802) diluted at 1/400 in PBS (PAN, Batch: 1870415) containing 1% of foetal calf serum (FCS) and 0.1% of saponin. This antibody specific to the cell bodies and neurites, enables the death of the neuronal cells as well as the effect of the extract on the neurite growth to be studied. This antibody is developed by an anti-mouse goat IgG labelled with an Alexa Fluor 488 (Molecular Probe, Batch: 1664729) at a 1/400 dilution in PBS containing 1% of FCS, 0.1% of saponin, for 1 hour at room temperature.

The extract is tested for one culture. For each extract concentration and controls, 6 wells are evaluated, 30 photographs are taken per plate using ImageXpress (Molecular Devices) with a ×20 magnification, to evaluate the neuritic network and the neurone number. The photograph analysis is made using Custom Module Editor (Molecular Devices). The results are shown in terms of number of positive cells or of neuritic network labelled by the MAP-2 label.

Each row of the plate is organised the following way:
no treatment (negative control)/0.1% of DMSO
Aβ1-42 peptide 20 μM/0.1% of DMSO
Aβ1-42 peptide 20 μM/Root extract (10 ng/mL)/0.1% of DMSO
Aβ1-42 peptide 20 μM/Root extract (100 ng/mL)/0.1% of DMSO
Aβ1-42 peptide 20 μM/Root extract (1 μg/mL)/0.1% of DMSO
Aβ1-42 peptide 20 μM/Root extract (10 μg/mL)/0.1% of DMSO

Each row is repeated 6 times.

Statistical Analysis

All the data are expressed as a percent of the condition control (control=100%). All the values are expressed as an average+/−standard error of the mean (SEM) (s.e.mean) of n=6 wells per condition. The statistical analyses made for the different conditions are analyses of variances or ANOVA followed by the Fischer PLSD test or Dunnett test using the software GraphPad Prism.

### 5.2. Results

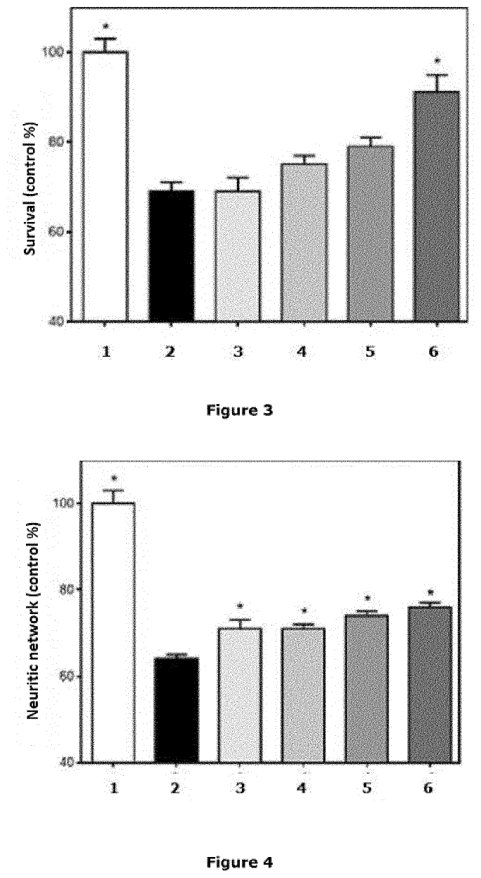

FIG. 3 represents the effect of the root extract from Tagetes erecta on the survival of primary cortical neurons intoxicated for 24 hours by the Aβ1-42 peptide added at a final concentration of 20 μM.

FIG. 4 represents the effect of the root extract from Tagetes erecta on the growth of the neuritic network intoxicated for 24 hours by the Aβ1-42 peptide added at a final concentration of 20 μM. These data make it possible to check the health status of the cortical neurons, because the stress generated by the Alzheimer's pathology through the Aβ1-42 peptide affects the number of neuron connections (neurites).

The control has not been treated with the Aβ1-42 peptide. Only the vehicle used for the root extract (the culture medium containing 0.1% of DMSO) has been added to the cell culture of cortical neurons. 100% neuron survival are observed under this condition.

Adding the Aβ1-42 peptide at a final concentration of 20 μM, induces at the end of 24 hours a significant neurone death (FIG. 3) as well as a significant loss of the neuritic network (FIG. 4) as previously indicated in literature (Callizot et al., 2013).

The root extract pre-incubated with the neuronal cells cultured 1 hour before adding the Aβ1-42 peptide brings about an effect on the neuronal survival increasingly important with increasing extract concentrations. The root extract at the highest concentration (10 μg/mL) shows a significant effect on the neuron survival (by 30%). The protective effect thus seems to depend on the dose applied: the effect increases with the dose applied (FIG. 3).

The root extract pre-incubated 1 hour with the neuronal cells cultured before adding the Aβ1-42 peptide shows a significant effect on the neuritic network from its lowest concentration of 10 ng/mL (FIG. 4).

These results show that the root extract from Tagetes erecta enables the neurotoxicity caused by the Aβ peptide to be reduced. This extract according to the invention is a good candidate for the protection of cortical neurons and the maintenance of their connections, that is maintenance of the integrity of the neuritic network. Thus, the extract according to the invention has an interest as a new therapeutic agent for preventing and/or treating Alzheimer disease.

1-10. (canceled)

11. A plant extract from the genus Tagetes enriched with leontopodic acid B and comprising at least 2.5% by weight of leontopodic acid B, expressed relative to the total weight of the dry extract.

12. The plant extract according to claim 11, comprising at least 5.5% by weight of leontopodic acid B.

13. The plant extract according to claim 11, comprising at least 8% by weight of leontopodic acid B.

14. The extract according to claim 11, wherein said plant from the genus Tagetes is chosen from the group consisting of Tagetes erecta and Tagetes patula.

15. A process for preparing a plant extract from the genus Tagetes according to claim 11, comprising:
a) a step of culturing Tagetes under soil-less conditions,
b) a step of solid/liquid extraction of the roots, and
c) a step of recovering the extract obtained during step b).

16. The process according to claim 15, wherein said step a) of culturing Tagetes under soil-less conditions takes place in aeroponics.

17. The process according to claim 15, wherein step b) of solid/liquid extraction of the roots comprises a first phase of bringing the roots still attached to the plant in contact with a solvent, followed by a second phase comprising severing said roots and macerating the severed roots in a solvent, said solvent, identical or different in the first and the second phase, being chosen from alcohols, glycols and eutectic solvents, and being used pure or in the form of an aqueous solution of alcohol, glycol or eutectic solvent.

18. The process according to claim 17, wherein said first phase of step b) of solid/liquid extraction of the roots has a duration between 15 minutes and 30 minutes.

19. The process according to claim 15, comprising:
a) a step of culturing Tagetes under soil-less conditions,
b) a step of stimulating the plant roots,
c) a step of solid/liquid extraction of the stimulated roots, and
d) recovering the extract obtained during step c).

20. A process according to claim 18, wherein said step a) of culturing Tagetes under soil-less conditions takes place in aeroponics.

21. The process according to claim 19, wherein step c) of solid/liquid extraction of the stimulated roots comprises a first phase of bringing the roots still attached to the plant in contact with a solvent, followed by a second phase comprising severing said roots and macerating the severed roots in a solvent, said solvent, identical or different in the first and the second phase, being chosen from alcohols, glycols and eutectic solvents, and being used pure or in the form of an aqueous solution of alcohol, glycol or eutectic solvent.

22. The process according to claim 21, wherein said first phase of step b) of stimulating the plant roots has a duration between 15 minutes and 30 minutes.

23. A cosmetic composition comprising, as an active agent, at least one extract according to claim 11 and at least one excipient.

24. The extract according to claim 11 for preventing or slowing skin ageing, and/or having a skin soothing or calming effect following the irritation feeling and/or stimulating epidermal turnover as well as the epidermal barrier function and skin moisturising and/or protecting the epidermis from environmental stresses.

25. A pharmaceutical composition or nutraceutical composition comprising, as an active agent, at least one extract according to claim 11 and at least one pharmaceutically or nutraceutically acceptable excipient.

26. The extract according to claim 11 for use as a drug for preventing or treating a neurodegenerative disease, including Alzheimer disease.

27. The extract according to claim 11 for use as a drug for stimulating antimicrobial defence and/or for mitigating or treating skin inflammation and/or for promoting skin healing, including in the case of a wound closure.

28. A skin cosmetic care method for preventing or delaying the onset of skin ageing effects, and/or having a skin soothing or calming effect following the irritation feeling and/or stimulating epidermal turnover as well as the epidermal barrier function and skin moisturising, and/or for protecting the epidermis from environmental stresses, said method comprising applying, on at least one part of the body or face skin, a cosmetic composition according to claim 23.
